SEQUENCE LISTING (9 sequences)

SEQ ID NO: 1 - DNA, 3217 bp, Homo sapiens
ggccgggagacctggcggagctgggggtggggggccagtttttgcaacggctaaggaagggcctgtgggtttattataaggcggagctcggcgggagaggtgcgggccgaatccgagccgagcggagaggaatccggcagtagagagcggactccagccggcggaccctgcagccctcgcctgggacagcggcgcgctgggcaggcgcccaagagagcatcgagcagcggaacccgcgaagccggcccgcagccgcgacccgcgcagcctgccgctctcccgccgccggtccgggcagcatgaggcgcgcggcgctctggctctggctgtgcgcgctggcgctgagcctgcagccggccctgccgcaaattgtggctactaatttgccccctgaagatcaagatggctctggggatgactctgacaacttctccggctcaggtgcaggtgctttgcaagatatcaccttgtcacagcagaccccctccacttggaaggacacgcagctcctgacggctattcccacgtctccagaacccaccggcctggaggctacagctgcctccacctccaccctgccggctggagaggggcccaaggagggagaggctgtagtcctgccagaagtggagcctggcctcaccgcccgggagcaggaggccaccccccgacccagggagaccacacagctcccgaccactcatcaggcctcaacgaccacagccaccacggcccaggagcccgccacctcccacccccacagggacatgcagcctggccaccatgagacctcaacccctgcaggacccagccaagctgaccttcacactccccacacagaggatggaggtccttctgccaccgagagggctgctgaggatggagcctccagtcagctcccagcagcagagggctctggggagcaggacttcacctttgaaacctcgggggagaatacggctgtagtggccgtggagcctgaccgccggaaccagtccccagtggatcagggggccacgggggcctcacagggcctcctggacaggaaagaggtgctgggaggggtcattgccggaggcctcgtggggctcatctttgctgtgtgcctggtgggtttcatgctgtaccgcatgaagaagaaggacgaaggcagctactccttggaggagccgaaacaagccaacggcggggcctaccagaagcccaccaaacaggaggaattctatgcctgacgcgggagccatgcgccccctccgccctgccactcactaggcccccacttgcctcttccttgaagaactgcaggccctggcctcccctgccaccaggccacctccccagcattccagcccctctggtcgctcctgcccacggagtcgtggggtgtgctgggagctccactctgcttctctgacttctgcctggagacttagggcaccaggggtttctcgcataggacctttccaccacagccagcacctggcatcgcaccattctgactcggtttctccaaactgaagcagcctctccccaggtccagctctggaggggagggggatccgactgctttggacctaaatggcctcatgtggctggaagatcctgcgggtggggcttggggctcacacacctgtagcacttactggtaggaccaagcatcttgggggggtggccgctgagtggcaggggacaggagtccactttgtttcgtggggaggtctaatctagatatcgacttgtttttgcacatgtttcctctagttctttgttcatagcccagtagaccttgttacttctgaggtaagttaagtaagttgattcggtatccccccatcttgcttccctaatctatggtcgggagacagcatcagggttaagaagactttttttttttttttttaaactaggagaaccaaatctggaagccaaaatgtaggcttagtttgtgtgttgtctcttgagtttgtcgctcatgtgtgcaacagggtatggactatctgtctggtggccccgtttctggtggtctgttggcaggctggccagtccaggctgccgtggggccgccgcctctttcaagcagtcgtgcctgtgtccatgcgctcagggccatgctgaggcctgggccgctgccacgttggagaagcccgtgtgagaagtgaatgctgggactcagccttcagacagagaggactgtagggagggcggcaggggcctggagatcctcctgcagaccacgcccgtcctgcctgtggcgccgtctccaggggctgcttcctcctggaaattgacgaggggtgtcttgggcagagctggctctgagcgcctccatccaaggccaggttctccgttagctcctgtggccccaccctgggccctgggctggaatcaggaatattttccaaagagtgatagtcttttgcttttggcaaaactctacttaatccaatgggtttttccctgtacagtagattttccaaatgtaataaactttaatataaagtagtcctgtgaatgccactgccttcgcttcttgcctctgtgctgtgtgtgacgtgaccggacttttctgcaaacaccaacatgttgggaaacttggctcgaatctctgtgccttcgtctttcccatggggagggattctggttccagggtccctctgtgtatttgcttttttgttttggctgaaattctcctggaggtcggtaggttcagccaaggttttataaggctgatgtcaatttctgtgttgccaagctccaagccccatcttctaaatggcaaaggaaggtggatggccccagcacagcttgacctgaggctgtggtcacagcggaggtgtggagccgaggcctaccccgcagacaccttggacatcctcctcccacccggctgcagaggccagaggcccccagcccagggctcctgcacttacttgcttatttgacaacgtttcagcgactccgttggccactccgagaggtgggccagtctgtggatcagagatgcaccaccaagccaagggaacctgtgtccggtattcgatactgcgactttctgcctggagtgtatgactgcacatgactcgggggtggggaaaggggtcggctgaccatgctcatctgctggtccgtgggacggtgcccaagccagaggctgggttcatttgtgtaacgacaataaacggtacttgtcatttcgggcaaaaaaaaaaaaaaaaaa

SEQ ID NO: 2 - protein, 310 aa, Homo sapiens
MRRAALWLWLCALALSLQPALPQIVATNLPPEDQDGSGDDSDNFSGSGAGALQDITLSQQTPSTWKDTQLLTAIPTSPEPTGLEATAASTSTLPAGEGPKEGEAVVLPEVEPGLTAREQEATPRPRETTQLPTTHQASTTTATTAQEPATSHPHRDMQPGHHETSTPAGPSQADLHTPHTEDGGPSATERAAEDGASSQLPAAEGSGEQDFTFETSGENTAVVAVEPDRRNQSPVDQGATGASQGLLDRKEVLGGVIAGGLVGLIFAVCLVGFMLYRMKKKDEGSYSLEEPKQANGGAYQKPTKQEEFYA

SEQ ID NO: 3 - DNA, 102 bp, Homo sapiens
ctgcctccacctccaccctgccggctggagaggggcccaaggagggagaggctgtagtcctgccagaagtggagcctggcctcaccgcccgggagcaggagg

SEQ ID NO: 4 - protein, 34 aa, Homo sapiens
ASTSTLPAGEGPKEGEAVVLPEVEPGLTAREQEA

SEQ ID NO: 5 - DNA, 20 bp, Artificial Sequence
  Description of Artificial Sequence  Synthetic Primer
aggacttcacctttgaaacc

SEQ ID NO: 6 - DNA, 20 bp, Artificial Sequence
  Description of Artificial Sequence  Synthetic Primer
aggaggaattctatgcctga

SEQ ID NO: 7 - DNA, 19 bp, Artificial Sequence
  Description of Artificial Sequence  Synthetic Primer
ggtaagttaagtaagttga

SEQ ID NO: 8 - protein, 234 aa, Homo sapiens
QPALPQIVATNLPPEDQDGSGDDSDNFSGSGAGALQDITLSQQTPSTWKDTQLLTAIPTSPEPTGLEATAASTSTLPAGEGPKEGEAVVLPEVEPGLTAREQEATPRPRETTQLPTTHQASTTTATTAQEPATSHPHRDMQPGHHETSTPAGPSQADLHTPHTEDGGPSATERAAEDGASSQLPAAEGSGEQDFTFETSGENTAVVAVEPDRRNQSPVDQGATGASQGLLDRKE

SEQ ID NO: 9 - protein, 293 aa, Homo sapiens
QPALPQIVATNLPPEDQDGSGDDSDNFSGSGAGALQDITLSQQTPSTWKDTQLLTAIPTSPEPTGLEATAASTSTLPAGEGPKEGEAVVLPEVEPGLTAREQEATPRPRETTQLPTTHQASTTTATTAQEPATSHPHRDMQPGHHETSTPAGPSQADLHTPHTEDGGPSATERAAEDGASSQLPAAEGSGEQDFTFETSGENTAVVAVEPDRRNQSPVDQGATGASQGLLDRKEVLGGVIAGGLVGLIFAVCLVGFMLYRMKKKDEGSYSLEEPKQANGGAYQKPTKQEEFYA

(19) United States
(12) Patent Application Publication
(10) Pub. No.: US 2006/0134122 A1
(43) Pub. Date: Jun. 22, 2006

(54) Peptides of syndecan-1 for inhibition of cancer

(76) Inventors: Alan C. Rapraeger, Madison, WI (US); DeannaLee M. Beauvais, Madison, WI (US)

Correspondence Address: FULBRIGHT & JAWORSKI L.L.P., 600 CONGRESS AVE., SUITE 2400, AUSTIN, TX, 78701, US

(21) Appl. No.: 11/291,112

(22) Filed: Nov. 30, 2005

Related U.S. Application Data
(60) Provisional application No. 60/636,685, filed on Dec. 16, 2004.

(51) Int. Cl. A61K 39/395; C07H 21/04; C12P 21/06; C12N 5/06; C07K 16/30

(52) U.S. Cl. 424/155.1; 435/69.1; 435/320.1; 435/338; 530/388.8; 536/23.53

(57) ABSTRACT

The present invention provides a novel peptide from the extracellular domain of syndecan-1 that inhibits migration, metastasis, survival and proliferation of cancer cells.

The present application claims benefit of priority to U.S. Provisional Application Ser. No. 60/636,685, filed Dec. 16, 2005, the entire contents of which are hereby incorporated by reference.

The government may own rights to the present invention pursuant to the National Institute of Health grant numbers R01-HD21881 and CA109010.

### BACKGROUND OF THE INVENTION

I. Field of the Invention

The present invention relates generally to the fields of protein chemistry and cancer biology. More particularly, it concerns peptide segments from the extracellular domain of syndecan-1 (Sdc-1) that can inhibit cancers, particularly various carcinomas, and prevent angiogenesis associated with tumor growth, diabetic retinopathy and other associated diseases.

II. Description of Related Art

A. αᵥβ₃ and αᵥβ₅ Integrins

The αᵥβ₃ and αᵥβ₃ are closely related integrins that are upregulated during disease processes. The αᵥβ₃integrin is a key regulator of adhesion and signaling in numerous biological processes, including tumor cell migration and metastasis, and angiogenesis. The activated form of this integrin participates in arrest of tumor cells in the blood stream (Pilch et al., 2002), enhancing their extravasation to target tissues, especially bone, where the activated integrin has further roles in tumor cell proliferation and survival (Brooks et al., 1994; Petitclerc et al., 1999; Eliceiri, 2001). In endothelial cells forming new blood vessels, the active integrin is linked not only to adhesion-dependent processes but also to signaling in response to FGF-2 (Eliceiri et al., 1998; Hood et al., 2003).

Although αᵥβ₃integrin expression in mammary epithelium is low, activated αᵥβ₃is expressed on most, if not all, successfull mammary carcinoma metastases (Liapis et al., 1996; Felding-Habermann et al., 2001). The inventors have reported previously that a° P3 integrin on MDA-MB-231 mammary carcinoma cells appears to be functionally linked to Sdc-1; the cells spread when adherent to an artificial substratum comprised solely of Sdc1-specific antibody, and although this spreading occurs in the absence of an αᵥβ₃ ligand, the spreading requires activated αᵥβ₃ integrin (Beauvais and Rapraeger, 2003). This finding suggests that even on a native ECM, anchorage of Sdc-1 to the matrix may serve as an important regulator of αᵥβ₃ integrin activation and signaling.

Although classically defined as a vitronectin (VN) receptor, αᵥβ₃ is promiscuous and binds many ECM components including fibronectin (FN), fibrinogen, von Willebrand Factor, proteolysed fragments of collagen (COL), laminin (LN), osteopontin, and others (van der Flier and Sonnenberg, 2001). Mechanisms leading to activation of this integrin are complex, including proteolytic cleavage (Ratnikov et al., 2002), conformational changes (affinity modulation), and clustering (avidity modulation; Carman and Springer, 2003). Activation is regulated by “inside-out” signals from the cell interior and is stabilized by ligand interactions that trigger “outside-in” signaling (Giancotti and Ruoslahti, 1999). Cell surface receptors known to modulate αᵥβ₃ activity include CD87/uPAR and CD47/IAP, which associate with the β₃ integrin subunit via their extracellular domains (Lindberg et al., 1996; Xue et al., 1997) and may also regulate αᵥβ₃ function indirectly via a pertussis toxin-sensitive G-protein signaling pathway (Gao et al., 1996; Degryse et al., 2001).

The αᵥβ₅ integrin is a close relative of the αᵥβ₃integrin. The αᵥβ₃ integrin is expressed on a variety of tissues and cell types, including endothelia, epithelia and fibroblasts (Felding-Habermann and Cheresh, 1993; Pasqualini et al., 1993). It is closely related to the αᵥβ₃integrin (56.1% identity and 83.5% homology between the two integrin β-subunits) but is distinguished from the αᵥβ₃by divergent sites near its ligand-binding domain and within the C-terminus of its cytoplasmic domain (McLean et al., 1990). It has a role in matrix adhesion to VN, FN, SPARC and bone sialoprotein (Plow et al., 2000) and is implicated in the invasion of gliomas and metastatic carcinoma cells (Brooks et al., 1997; Jones et al., 1997; Tonn et al., 1998), the latter especially to bone (De et al., 2003). A second major role is in endocytosis, including endocytosis of VN (Memmo and McKeown-Longo, 1998; Panetti et al., 1995), the engulfrnent of apoptotic cells by phagocytes (Albert et al., 2000), and participation in the internalization of shed outer rod segments in the retinal pigmented epithelium (Finnemann, 2003a; Finnemann, 2003b; Hall et al., 2003). A third major role is in growth factor-induced angiogenesis, where cooperative signaling by the αᵥβ₅ integrin and growth factors regulates endothelial cell proliferation and survival. Angiogenesis promoted by VEGF and TGFα in human umbilical vein endothelial cells relies on co-signaling with the αᵥβ₅ integrin, whereas FGF-2 and tumor necrosis factor-α collaborate with the αᵥβ₃ integrin (Eliceiri and Cheresh, 1999; Friedlander et al., 1995).

B. Syndecans

The syndecan family of cell surface heparan sulfate (HS) proteoglycans is comprised of four vertebrate members. These receptors are expressed on virtually all cell types, although their expression may be altered in disease states such as cancer (Beauvais and Rapraeger, 2004). The syndecan core proteins share a high degree of conservation in their short cytoplasmic and transmembrane (TM) domains; in contrast, their ectodomains (EDs) are divergent with the exception of attachment sites for HS glycosaminoglycans. Via their HS chains, syndecans regulate the signaling of growth factors, chemokines, and morphogens and engage components of the ECM including VN, FN, LN, tenascin, thrombospondin, and the fibrillar COLs (Bernfield et al., 1999).

In addition to the activities of their HS chains, the syndecan core proteins have roles in cell adhesion signaling (Rapraeger, 2000; Tumova et al., 2000). Conserved and variable regions of the syndecan cytoplasmic domains appear critical for binding interactions that lead to adhesion-mediated signaling and reorganization of the actin cytoskeleton (Couchman et al., 2001). Important roles for the TM domain have also been demonstrated for Sdc-1 and syndecan-4 (Sdc-4) (Tkachenko and Simons, 2002; McQuade and Rapraeger, 2003). Perhaps the least expected active protein domain is the syndecan ED, which bears the HS chains. Nonetheless, several emerging studies suggest that the syndecan ED may have important regulatory roles in cell adhesion signaling. Cell spreading and morphogenetic activities in COS-7 and Schwann cells trace in part to the S1ED (Carey et al., 1994; Adams et al., 2001). Raji cells require the Sdc-1 TM domain for initial spreading, but depend on a S1ED activity for cell polarization (McQuade and Rapraeger, 2003). Moreover, inhibition of ARH-77 myeloma and hepatocellular carcinoma cell invasion into a COL I matrix by Sdc-1 also traces to a region of its extracellular core protein domain (Liu et al., 1998; Ohtake et al., 1999).

The activities of other syndecans also trace to their EDs. Overexpression of Syndecan-2 (Sdc-2) in COS-1 and Swiss 3T3 cells induces filipodial extension and deletion mutants of Sdc-2 map activity to the S2ED (Granes et al., 1999). Upregulation of Sdc-2 expression in colon carcinoma cells leads to altered cell morphology and colony formation in soft agar; treatment with recombinant S2ED disrupts these behaviors (Park et al., 2002; Kim et al., 2003). Finally, activated B-lymphocytes, when seeded on S4ED antibodies, exhibit morphological changes and filipodial extensions. Intriguingly, only the S4ED is required for this response, indicating that it may interact with a TM partner to transmit a dendritic signal (Yamashita et al., 1999).

C. αᵥβ₃ Integrins are Regulated by Syndecan-1

The inventors' previous work in the MDA-MB-231 cells suggested that cell spreading induced upon anchorage of the cells to a Sdc-1 antibody relies on functional coupling of the syndecan to activated αᵥβ₃ integrins (Beauvais and Rapraeger, 2003). This spreading response is rapid (˜15-30 min) and occurs even in the absence of an integrin ligand (i.e., spreading is not blocked by cycloheximide or EGTA treatment), so long as the cells are adherent via Sdc-1. Intriguingly, the αᵥβ₃-dependent spreading mechanism is blocked by the addition of soluble, recombinant S1ED, suggesting that anchorage of Sdc-1 to a ligand provides a platform for αᵥβ₃ integrin activation and adhesion signaling via binding interaction of its ED. These findings raised a fundamental question about the role of Sdc-1 in ECM signaling, in particular whether or not Sdc-1 is required for αᵥβ₃ activation and signaling in response to a native matrix ligand.

### SUMMARY OF THE INVENTION

Thus, in accordance with the present invention, there is provided an isolated and purified peptide or polypeptide segment consisting of 5-240 amino acid residues and comprising at least 5 contiguous residues of SEQ ID NO:4. The peptide may be 5, 10, 15, 20, 25, 30, 34, 40, 50, 60, 70, 80, 90, 100, 110, 120, 130, 140, 150, 160, 170, 180, 190, 200, 210, 220, 230, 234 or 240 amino acid residues in length. The peptide or polypeptide may comprise 5, 10, 15, 20, 25, or 30 consecutive amino acid residues of SEQ ID NO:4, or the entire length of SEQ ID NO:4 or SEQ ID NO:8 or SEQ ID NO:9. The peptide or polypeptide may comprise 5, 10, 15, 20, 25, 30, 34, 40, 50, 60, 70, 80, 90, 100, 110, 120, 130, 140, 150, 160, 170, 180, 190, 200, 210, 220, 230, 234 or 240 consecutive amino acid residues of SEQ ID NOS:8 or 9, or may peptide consists of SEQ ID NOS:8 or9.

In another embodiment, there is provided a nucleic acid encoding an isolated and purified peptide or polypeptide consisting of 5-240 amino acid residues and comprising at least 5 contiguous residues of SEQ ID NO:4 or SEQ ID NO:8 or SEQ ID NO:9. The nucleic acid may encode a peptide or polypeptide of 5, 10, 15, 20, 25, 30, 34, 40, 50, 60, 70, 80, 90, 100, 110, 120, 130, 140, 150, 160, 170, 180, 190, 200, 210, 220, 230, 234 or 240 amino acid residues in length, in particular a peptide or polypeptide comprising 5, 10, 15, 20,25, 30, 34,40, 50, 60, 70, 80, 90, 100,110, 120, 130, 140, 150, 160, 170, 180, 190, 200, 210, 220, 230, 234 or 240 consecutive amino acid residues of SEQ ID NOS:4, 8 or 9, and specifically, a peptide or polypeptide consisting of SEQ ID NO:4 or SEQ ID NO:8 or SEQ ID NO:9.

In yet another embodiment, there is provided a recombinant cell comprising a nucleic acid encoding an peptide or polypeptide consisting of 5-240 amino acid residues and comprising at least 5 contiguous residues of SEQ ID NO:4 or SEQ ID NO:8 or SEQ ID NO:9, or any of the peptides or polypeptides described above. The cell may be a bacterial cell. The nucleic acid may further encode a peptide tag fused to the nucleic acid encoding the peptide segment.

In still yet another embodiment, there is provided a method of inhibiting interaction of αᵥβ₃ or αᵥβ₅ integrin with syndecan-1 comprising contacting a αᵥβ₃ or αᵥβ₅ integrin molecule with a peptide or polypeptide consisting of 5-240 amino acid residues and comprising at least 5 contiguous residues of SEQ ID NO:4 or SEQ ID NO:8 or SEQ ID NO:9. The peptide or polypeptide may be 5, 10, 15, 20, 25, 30, 34, 40, 50, 60, 70, 80,90, 100, 110, 120, 130, 140, 150, 160, 170, 180, 190, 200, 210, 220, 230, 234 or 240 amino acid residues in length, may comprise 5, 10, 15, 20, 25, 30, 34, 40, 50, 60, 70, 80, 90, 100, 110, 120, 130, 140, 150, 160, 170, 180, 190, 200, 210, 220, 230, 234 or 240 consecutive amino acid residues of SEQ ID NOS:4, 8 or 9, and in particular may consists of SEQ ID NO:4 or SEQ ID NO:8 or SEQ ED NO:9. The αᵥβ₃ or αᵥβ₅ integrin may be located on the surface of a cell.

In a further another embodiment, there is provided a method of inhibiting αᵥβ₃or αᵥβ₅ integrin activation by syndecan-I comprising contacting a cell expressing an αᵥβ₃ or αᵥβ₅ integrin molecule with a peptide or polypeptide consisting of 5-240 amino acid residues and comprising at least 5 contiguous residues of SEQ ID NO:4 or SEQ ID NO:8 or SEQ ID NO:9, or any of the peptides or polypeptides described above.

In still a further embodiment, there is provided a method of inhibiting a cancer cell expressing αᵥβ₃ or or αᵥβ₅ integrin comprising contacting a cell expressing an αᵥβ₃ or or αᵥβ₅ integrin molecule with a peptide or polypeptide consisting of 5-240 amino acid residues and comprising at least 5 contiguous residues of SEQ ID NO:4 or SEQ ID NO:8 or SEQ ID NO:9. The peptide or polypeptide may be 5, 10, 15, 20, 25, 30, 34, 40, 50, 60, 70, 80, 90, 100, 110, 120, 130, 140, 150, 160, 170, 180, 190, 200, 210, 220, 230, 234 or 240 amino acid residues in length, may comprise 5, 10, 15, 20, 25, 30, 34, 40, 50, 60, 70, 80, 90, 100, 110, 120, 130, 140, 150, 160, 170, 180, 190, 200, 210, 220, 230, 234 or 240 consecutive amino acid residues of SEQ ID NO:4 or SEQ ID NO:8 or SEQ ID NO:9, and in particular may consists of SEQ ID NO:4 or SEQ ED NO:8 or SEQ ID NO:9. Inhibiting may comprise inhibiting migration, metastasis, survival and/or proliferation. The cancer cell may be a carcinoma, a myeloma, a melanoma or a glioma. The method may further comprising contacting the cell with a second cancer inhibitory agent.

In still yet a further embodiment, there is provided a method of treating a subject with a cancer, cells of which express αᵥβ₃ or or αᵥβ₅ integrin, comprising contacting a cell expressing an αᵥβ₃ or or αᵥβ₅ integrin molecule with a peptide or polypeptide consisting of 5-240 amino acid residues and comprising at least 5 contiguous residues of SEQ ID NO:4 or SEQ ID NO:8 or SEQ ID NO:9. The peptide or polypeptide may be 5, 10, 15, 20, 25, 30, 34, 40, 50, 60, 70, 80, 90, 100, 110, 120, 130, 140, 150, 160, 170, 180, 190, 200, 210, 220, 230, 234 or 240 amino acid residues in length, may comprise 5, 10, 15, 20, 25, 30, 34, 40, 50, 60, 70, 80, 90, 100, 110, 120, 130, 140, 150, 160, 170, 180, 190, 200, 210, 220, 230, 234 or 240 consecutive amino acid residues of SEQ ID NO:4 or SEQ ID NO:8 or SEQ ID NO:9, and in particular may consist of SEQ ID NO:4 or SEQ ID NO:8 or SEQ ID NO:9. The subject may be a human. The cancer may be a carcinoma, a myeloma, a melanoma or a glioma. The peptide or polypeptide is administered directly to the cancer cells, local to the cancer cells, regional to the cancer cells, or systemically. The method may further comprise administering to the subject a second cancer therapy selected from chemotherapy, radiotherapy, immunotherapy, hormonal therapy, or gene therapy.

In an additional embodiment, there is provided a method of inhibiting angiogenesis comprising contacting an endothelial cell expressing an αᵥβ₃ or or αᵥβ₅ integrin molecule with a peptide or polypeptide consisting of 5-240 amino acid residues and comprising at least 5 contiguous residues of SEQ ID NO:4 or SEQ ID NO:8 or SEQ ID NO:9. The peptide or polypeptide may be 5, 10, 15, 20, 25, 30, 34, 40, 50, 60, 70, 80, 90, 100, 110, 120, 130, 140, 150, 160, 170, 180, 190, 200, 210, 220, 230, 234 or 240 amino acid residues in length, may comprise 5, 10, 15, 20, 25, 30, 34, 40, 50, 60, 70, 80, 90, 100, 110, 120, 130, 140, 150, 160, 170, 180, 190, 200, 210, 220, 230, 234 or 240 consecutive amino acid residues of SEQ ID NO:4 or SEQ ID NO:8 or SEQ ID NO:9, and in particular may consist of SEQ ID NO:4 or SEQ ID NO:8 or SEQ ID NO:9.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more, ” “at least one,” and “one or more than one.” The word “about” means plus or minus 5% of the stated number.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed.

FIG. 1 —MDA-MB-231 human mammary carcinoma cell spreading on VN, but not FN, is disrupted by soluble, recombinant Sdc-1ED. Cells were plated on wells coated with 10 μg/mL of VN or FN in plating medium alone or medium containing either 30 μg/mL mAb LM609, 25 μg/mL mAb 13, 20 μM GST-mS1ED or -mS4ED. Cells were incubated at 37° C. for 2 h, fixed and stained with rhodamine-conjugated phalloidin. Bar, 50 μm.

FIGS. 2A-C—Sdc-1 adhesion-mediated cell spreading correlates with αᵥβ₃ integrin. expression and activity. (FIG. 2A) FACS analysis of αᵥβ₃ integrin expression (mAb LM609) in human mammary carcinoma cells against an IgG isotype control. (FIG. 2B) Depicted on split panels are phalloidin-stained MDA-MB-435 (upper half) and MCF-7 (lower half) cells 2 h after plating on wells coated with mAb B-B4 in plating medium alone or medium containing either 30 μg/mL mAb LM609, 1 μg/mL mAb 13 or 20 μM GST-mS1ED. Bar, 50 μm. (FIG. 2C) Untreated MDA-MB-435, MDA-MB-231 and MCF-7 cells (columns 1 and 4) and cells pretreated in suspension with 1 μg/mL mAb 13 (columns 2 and 3) were seeded on wells coated with either mAb B-B4 or COL I in plating medium alone (columns 1, 2 and 4) or medium containing 20 μM GST-mS1ED (column 3). Cells were incubated at 37° C. for 2 h, fixed, permeabilized and stained with WOW1 and an Alexa 488-conjugated secondary. Panel insets are corresponding phase contrast pictures. Bar, 20 μm.

FIG. 3—MDA-MB-435, but not MCF-7, human carcinoma cells display functional coupling of Sdc-1 and αᵥβ₃ integrins on VN. Depicted are phalloidin-stained cells 2 h after plating on wells coated with 10 μg/mL VN (upper half) or FN (lower half) in plating medium alone or medium containing either 30 μg/mL mAb LM609, 25 μg/mL mAb 13, 20 μM GST-mS1ED or -mS4ED. Bar, 50 μm.

FIG. 4—Polyclonal Sdc-1 ED antibodies disrupt αᵥβ₃ integrin-dependent cell spreading on VN. MDA-MB-231 and -435 cells were plated on wells coated with 10 μg/mL of VN (upper half) or FN (lower half) in plating medium alone or medium containing 10, 100 or 250 μg/mL anti-mSdc-1ED pAb. Cells were incubated at 37° C. for 2 h, fixed and stained with rhodamine-conjugated phalloidin. Bar, 50 μm.

FIGS. 5A-B—Sdc-1ED Inhibitors disrupt αᵥβ₃ integrin-dependent cell migration on VN. MDA-MB-231, MDA-MB-435 and MCF-7 cells in plating medium alone (black-filled) or in plating medium containing either 20 μM GST-mS1ED (open) or 250 μg/mL polyclonal mS1ED antibodies (gray-filled) were seeded on polycarbonate filters coated with either 10 μg/ml of VN (FIG. 5A) or FN (FIG. 5B) in a modified Boyden chamber. After 16 h, cells that migrated through the filter in response to 10% FBS in the lower chamber were quantified by colorimetric staining.

FIGS. 6A-B—Deletion of a region of the Sdc-1ED blocks αᵥβ₃-mediated cell spreading. (FIG. 6A) Graphic representation of S1 expression constructs transfected into MDA-MB-231 cells and their relative expression levels as detected by FACS (mean fluorescent intensity). Note the locations of the mAb 281.2 and B-B4 binding sites; asterisks indicate the HS attachment sites. (FIG. 6B) Cells transfected with empty vector (NEO) or S1 constructs were seeded in plating medium on wells coated with either anti-hS1 mAb B-B4 (NEO+inset, hS1 and insets of mS1) or anti-mS1 mAb 281.2 (all others). Where noted, cells were pretreated in suspension with 1 μg/mL mAb P5D2 for 15 min prior to plating. Cells were incubated at 37° C. for 2 h, fixed and stained with rhodamine-conjugated phalloidin. Bar, 50 μm.

FIGS. 7A-F—Overexpression and ligation of Sdc-1 activates αᵥβ₃ integrins and “primes” cells to spread on VN. (FIG. 7A) MDA-MB-231 cells transfected with empty vector (NEO), GPI-mS1ED or mS1^{TDM} were seeded on wells coated with 1, 3, or 10 μg/mL VN. Cells were incubated at 37° C. for 2 h, fixed and stained with rhodamine-conjugated phalloidin. Bar, 50 μm. (FIGS. 7B-E) Suspended cells in which mS1 was clustered (mAb 281.2, FIG. 7B and 7E) or not clustered (mAb KY8.2, FIGS. 7C and 7D) in plating medium were fixed and labeled with either mAb LM609 (FIG. 7B) or WOW1 mouse Fab (FIGS. 7C-E) followed by an Alexa 488-conjugated secondary and analyzed by FACS. As controls for WOW1 staining (FIG. 7C), suspended cells were incubated with plating medium alone (Dlack-filled histogram), plating medium containing 1 mM MnCl₂ (right-shifted histogram) or divalent cation-free PBS (left shifted histogram) prior to fixation and staining. (FIG. 7F) Cells were seeded on wells coated with either mAb B-B4 or 281.2, incubated at 37° C. for 2 h, fixed, permeabilized and stained with WOW1 and an Alexa 488-conjugated secondary. Panel insets are corresponding phase contrast pictures. Bar, 20 μm.

FIGS. 8A-H—Downregulation of Sdc-1 expression by siRNA disrupts cell spreading and migration on VN. (FIG. 8A) SiRNA targeting of hS1 mRNA. FACS analysis for (FIGS. 8B-C) hS1 (mAb B-B4), (FIG. 8D) hS4 (mAb F94-8G3) and (FIG. 8E) mS1 (mAb 281.2) expression against IgG controls (black filled histograms) in NEO and GPI-mS1ED expressing MDA-MB-231 cells 72 h after transfection with either lipid-vehicle alone (Vehicle or (V)) or 200 nM siRNA (RNAi or (R)). (FIG. 8F) NEO^{MDA}-MB-231 cells and MDA-MB-231 cells expressing GPI-mS1ED, mS1^{Δ88-252}, mS1^{Δ122-252} and mS1^{TDM} were transfected with lipid-vehicle alone or 200 nM hS1-siRNA and seeded on wells coated with either 10 μg/ml of VN or FN. Cells were incubated at 37° C. for 2 h, fixed and stained with rhodamine-conjugated phalloidin. Bar, 50 μm. (FIG. 8G-H) Lipid-vehicle (gray-filled) or hS1-siRNA (black-filled) transfected cells were also plated on polycarbonate filters coated with either 10 μg/ml of VN (FIG. 8G) or FN (FIG. 8H) in a modified Boyden chamber. After 16 h, cells that migrated through the filter in response to 10% FBS in the lower chamber were quantified by colorimetric staining.

FIG. 9—VN induces complete spreading of B82L fibroblasts. B82L cells were plated on wells co-coated with increasing amounts of VN and 60 μg/mL mAb 281.2, 150 μg/mL S4ED pAb or in the absence of antibody. Cells were allowed to spread 2 h before fixation and labeling with rhodamine-phalloidin.

FIGS. 10A-D—siRNA blockade of β₅ subunit expression blocks syndecan-induced cell spreading. (FIG. 10A) Suspended cells are analyzed by flow cytometry using antibodies capable of recognizing mouse β₁ (HMβ1-1), β₃ (2C9.G2) or αᵥ (H9.2B8) integrin subunits, mAb 281.2 specific for mouse syndecan-1, or nonspecific IgG control (gray fill). Cells treated with β₅-integrin-specific or control siRNA are compared. (FIG. 10B) Representative western blot of lysates of cells treated with 0, 200, 400, 600 or 800 nM β₅-specific siRNA and probed for expression of β₅ integrin subunit. FAK expression levels are shown as a loading control. (FIG. 10C) Quantification (±S.E.) of relative β₅ integrin subunit expression from duplicate blots as described in (FIG. 10B). (FIG. 10D) B82L cells were plated on wells coated with 60 μg/mL mAb 281.2 and increasing amounts of VN after treatment with β₅-integrin-specific or control siRNA. Cells were allowed to spread 2 h before fixation and labeling with rhodamine-phalloidin.

FIGS. 11A-F—Downregulation of mouse Sdc-1 expression by siRNA blocks αᵥβ₅-dependent cell attachment and spreading on VN. FACS analysis for (FIG. 11A) mouse syndecan-1 (mAb 281.2), (FIG. 11B) αᵥ integrin subunit (mAb H9.2B8), (FIG. 11C) mouse Syndecan-4 (mAb KY8.2), (FIG. 11D) human syndecan-1 (mAb B-B4) and (FIG. 11E) FcR^{ecto}-hS1 chimera (FITC-conjugated hIgG) expression against isotype IgG controls (red-fill) in vector^{NEO} (FIGS. 11A-C), human syndecan-1 (FIG. 11D) and FcR^{ecto}-hS1 (FIG. 11E) expressing B82L cells 48 h after transfection with either 600 nM control (Control) or mouse syndecan-1-specific siRNA (siRNA). (FIG. 11F) B82L^{NEO} empty vector-transfected control cells and B82L cells expressing human syndecan-1 or the FcR^{ecto}-hS1 chimera were transfected with control or mouse syndecan-1-specific siRNA and seeded on wells coated with either 5 μg/mL VN alone or a mixed substratum of VN plus 60 μg/mL of antibody directed against mouse syndecan-1 (281.2), human syndecan-1 (B-B4) or the FcR^{ecto}-hS1 chimera (hIgG). Cells were incubated at 37° C. for 2 h, fixed, and stained with rhodamine-conjugated phalloidin.

FIGS. 12A-B—B82L cell spreading on VN is blocked by recombinant Scd-1ED. (FIG. 12A) B82L fibroblasts were plated on 5 μg/mL VN in the absence of other treatment, or in the presence of 30 μM GST, 1-30 μM GST-mS1ED or 30 μM GST-mS4ED (inset), then fixed and stained with rhodamine-phalloidin for visualization. (FIG. 12B) Quantification of cell adhesion in triplicate samples (±S.E.) plated either with no additions, or concentrations of GST-mS1ED ranging from 0-30 μM.

FIG. 13—Human aortic endothelial cells rely on αᵥβ₃ and αᵥβ₅ integrins for spreading on VN. Human aortic endothelial cells (HAECs) were suspended and replated on 10 μg/ml VN in the presence of integrin inhibitory antibodies mAb 13 (specific for β1-containing integrins; this will inhibit the αᵥβ1 integrin), LM609 (specific for the αᵥβ3 integrin) and P1F6 (specific for the αᵥβ5 integrin), or combinations of these antibodies. After 2 hr, the cells were fixed, stained with Alexa488-conjugated phalloidin to aid in visualization, and observed by fluorescence microscopy to document integrin-dependent spreading. Inhibition of spreading requires simultaneous inhibition of both the αᵥβ3 and αᵥβ5 integrins.

FIGS. 14A-C—Co-expression of Sdc-1, αᵥβ₃ integrin and αᵥβ₅ integrin by endothelial cells during tumor-induced angiogenesis. (FIG. 14A) Wnt-1 induced mammary tumor; (FIG. 14B) β-catenin-induced mammary tumor; (FIG. 14C) Normal mouse artery. Endothelial cells are identified by staining with a rat monoclonal for PECAM. The integrin subunits are identified by staining with rabbit polyclonal antibodies to the β3 subunit (AB1932), β₅ subunit (AB1926) and αV subunit (AB1930). Sdc-1 is visualized by an affinity purified rabbit polyclonal antibody directed against its extracellular domain. Rat IgG and Rabbit IgG controls are shown. The staining in the artery is shown as an overlay of the two antibodies used.

FIG. 15—αᵥβ3 and αᵥβ5 integrin-mediated spreading of human aortic endothelial cells on VN is disrupted by recombinant syndecan-1 ectodomain fusion protein. HAECs are plated on VN in the presence of no competitor, 1-10 uM GST-mS1ED (recombinant mouse syndecan-1 ectodomain expressed as a fusion protein with GST) or GST alone.

FIG. 16—Inhibition of HAEC spreading on VN by silencing of Sdc-1. HAECs were transfected with a range of concentrations of siRNA specific for human Sdc1 (hS1), which achieves 80-88% silencing at 100-200 nM, as shown by flow cytometry using human Sdc-1 mAb B-B4. The cells treated with 200 nM siRNA were also plated on VN for 2 hr and display a 60% reduction in cell spreading.

FIGS. 17A-B—Competition with soluble Sdc-1 ectodomain causes enhanced apoptosis of human microvascular endothelial cells (HMECs) in the presence of FGF. (FIG. 17A) HMEC-1 cells were plated overnight on gelatin, then starved for 24 hr to induce the intrinsic pathway of apoptosis. Apoptotic cells were easily observed by DIC microscopy. Examples of apoptotic cells are denoted by black arrows, living cells by red arrows. Cells were treated with 100 ng/ml VEGF, 100 ng/ml FGF, FGF+30 ug/ml hScd-1ED or hScd-1ED alone. Control cells in 15% serum showed little or no cell death, and hScd-1ED in 15% serum had no effect (not shown). (FIG. 17B) Quantification of % protection from apoptosis by each treatment. Note that hScd-1ED actually has a protective effect when added alone, but dramatically increases apoptosis in the presence of FGF, consistent with the FGF stimulation requiring simultaneous signaling from the αᵥβ3 integrin, which is inhibited by hScd-1ED. VEGF has no effect on this pathway, as it affects the extrinsic pathway of apoptosis which is not induced by serum starvation.

### DESCRIPTION OF ILLUSTRATIVE EMBODIMENTS

### I. THE PRESENT INVENTION

As discussed above, the inventors have established a link between Sdc-1 and the αᵥβ₃. The inventors now report that MDA-MB-231 and MDA-MB-435 human mammary carcinoma cells, which express αᵥβ₃, require Sdc-1 engagement with the matrix for αᵥβ₃ activity on VN. Addition of recombinant mouse Sdc-1 (mS1) ED or anti-Scd-1ED polyclonal antibodies (pAbs) block integrin activation and disrupt αᵥβ₃-dependent spreading and migration of MDA-MB-231 and -435 cells. In contrast, αᵥβ₁-dependent spreading and migration on FN is unaffected by these treatments. Furthermore, they also show that downregulation of human Sdc-1 (hS1) expression by small-interfering RNA (siRNA) disrupts MDA-MB-231 cell spreading and migration on VN, but not on FN. Expression of a mS1 construct containing the mS1 ED alone tethered to the membrane by a glycosylphosphatidylinositol (GPI) tail, which is unaffected by the human-specific siRNA, rescues αᵥβ₃-dependent spreading and migration on VN. These data suggest that Sdc-1 and the αᵥβ₃ integrin are functionally coupled via the S1ED and that coupling is required for αᵥβ₃ integrin activation and signaling.

Whether this functional coupling is via a signaling pathway, or via a direct interaction between these two receptors is not yet known; however, several features of this mechanism can be described. First, Sdc-1-dependent activation of αᵥβ₃ is contingent on the syndecan engaging a ligand. On antibody, ligation of Sdc-1 devoid of HS leads to integrin activation and cell spreading, the latter utilizing integrin-mediated signaling in the absence of an integrin ligand. On VN, a ligand that engages both Sdc-1 and αᵥβ₃, the integrin activation requires Sdc-1 bearing its HS chains, presumably to engage the heparin-binding domain of VN.

The finding that ligation of Sdc-1 is not only necessary but seemingly sufficient for integrin activation, as occurs on Sdc-1 antibody, is surprising. Most, if not all, ECM ligands have heparin binding domains that presumably engage Sdc-1; yet, the αᵥβ₃ integrin is not always active on these matrices. An example is the cell behaviors observed on FN, where αᵥβ₁ signaling predominates; the αᵥβ₃ integrin is inactive despite the fact that it (Charo et al., 1990) and Sdc-1 can engage the FN. Thus, unlike the antibody substratum, recognition of ECM components by other integrins and syndecans may disrupt the syndecan-αᵥβ₃ coupling mechanism or target the αᵥβ₃ directly, thus defeating Sdc-1 and inactivating the integrin.

In the MDA-MB-231 cells, the αᵥβ₃ integrin is maintained in an inactive state by negative cross-talk, apparently from the αᵥβ₁ integrin (Beauvais and Rapraeger, 2003). When cells are plated on Sdc-1 antibody, this cross-talk mechanism prevents αᵥβ₃ integrin activation; however, expression of higher Sdc-1 levels overrides the competing inhibition from the a2PI integrin as long as Sdc-1 is ligated. Overexpression of mS1 will not lead to integrin activation if the cells are adherent only via their endogenous hS1, indicating that hS1 and mS1 act independently and are unlikely to multimerize. A similar “priming” of integrin activation due to enhanced Sdc-1 expression drives αᵥβ₃-dependent adhesion and spreading on low concentrations of VN—levels at which parental cells cannot respond. It is worth noting that increased Sdc-1 expression in breast carcinomas and melanomas correlates with an aggressive metastatic phenotype and poor clinical prognosis (Barbareschi et al., 2003; Burbach et al., 2003; Timar et al., 1992); this may trace to upregulation in αᵥβ₃ activity.

A second feature of the coupling mechanism is its reliance on the S1ED. Regardless of whether Sdc-1 is engaged by antibody or VN, integrin activation is blocked by treatments that target this domain, including competition with anti-Sdc-1 antibodies or recombinant S1ED. Furthermore, inactivation of the integrin seen upon siRNA-dependent inhibition of hS1 expression is overcome by expression of GPI-linked mS1ED. Admittedly, the inhibitory effects of these reagents on cell spreading and migration on VN was a surprise. Unlike the Sdc-1-antibody based adhesion assays, cells on VN are clearly provided an αᵥβ₃integrin ligand, yet even in the presence of this ligand, the integrin still requires Sdc-1 in order to signal indicating a potentially important role for the S1ED that extends beyond the initial activation of the integrin. Although further experimentation will be necessary to identify the active site, a syndecan mutant lacking amino acids 121-252 of the ED retains activity, whereas one lacking an additional 34 amino acids (Δ88-252) does not. Importantly, within this 34-amino acid stretch mS1 and hS1 share 58% identity and 72% homology, indicating that activity of the S1ED is likely conserved between the species. This is also evidenced by the fact that overexpression of either Sdc-1 species is sufficient to confer enhanced αᵥβ₃activity.

A third feature of the coupling mechanism is that it is specific for the αᵥβ₃ integrin. MCF-7 cells spread and migrate on VN, but utilize the αᵥβ₁ integrin. Sdc-1 is not required for the activity of this integrin, nor is it inactivated by any treatments that target Sdc-1. Similarly, cell spreading and migration on FN, which requires αᵥβ₁ integrin activity, appears to occur independent of Sdc-1. Vice versa, coupling to the αᵥβ₃ integrin appears to be specific for Sdc-1. Coupling is not observed in MDA-MB-231 cells adherent by Sdc-4 (i.e. treatment with mAb LM609 has no effect on cells adherent and spread on Sdc-4-antibody, mAb 150.9) nor in cells adherent to mAb RVS-10 (Chemicon), an anti-CD71/transferrin receptor antibody (unpublished data). CD71-adherent cells fail to spread even in the presence of a function-blocking β₁ integrin antibody that stimulates spreading in cells adherent via Sdc-1 (Beauvais and Rapraeger, 2003).

Multiple mechanisms, including affinity and avidity modulation, regulate integrin function. Affinity modulation of αᵥβ₃ is complex and involves conformational changes within its extracellular domain (Beglova et al., 2002; Takagi et al., 2002). This is regulated by “inside-out” signaling that impinges on the integrin's cytoplasmic domains either by stimulating proteolysis (Du et al., 1995; Smith, 1997), phosphorylation (Blystone et al., 1996; Jenkins et al., 1998) or binding of intracellular proteins such as talin (Calderwood et al., 2002; Calderwood et al., 1999) and β₃-endonexin (Kashiwagi et al., 1997; Shattil et al., 1995). These intracellular events lead to the exposure of ligand-binding epitopes in the integrin's extracellular domains (Hughes et al., 1996). Studies on VN suggest that αᵥβ₃ can assume two or more distinct activation states (Takagi et al., 2002) and distinct αᵥβ₃ conformations have been detected for different matrix ligands (Boettiger et al., 2001). Ligand binding, in turn, stabilizes structural changes that initiate “outside-in” signaling that include tyrosine phosphorylation of the β₃ cytoplasmic tail (Law et al., 1999; Schaffner-Reckinger et al., 1998) and association of the β₃ subunit cytoplasmic tail with intracellular effectors.

While interactions of αᵥβ₃ with extracellular ligands stimulate outside-in signaling, signaling via unligated αᵥβ₃ is important in a process known as “integrin-mediated death” (IMD) (Stupack et al., 2001). In cells sensitive to IMD, αᵥβ₃ may act as a sensor during cell invasion, inducing cell death when the cells encounter a non-permissive ECM (Ilic et al., 1998). Until now, it has been unclear whether unligated αᵥβ₃ integrins can participate in cell signaling processes other than IMD. However, in this study, the unligated αᵥβ₃is capable of transmitting signals that lead to cell spreading when Sdc-1 is engaged. It is appealing to speculate that Sdc-1 via its adhesion-dependent activation of αᵥβ₃ may act as a negative regulator of IMD. A corollary of this idea is that disrupting the link between Sdc-1 and the αᵥβ₃ integrin may spur IMD during such processes as angiogenesis of endothelial cells, which also rely upon this integrin (see below).

αᵥβ₃ integrins interact with a number of cell surface receptors including PDGFR-β (Borges et al., 2000; Schneller et al., 1997), VEGFR-2 (Borges et al., 2000; Soldi et al., 1999), CD87/uPAR (Wei et al., 1996) and CD47/IAP (Fujimoto et al., 2003; Lindberg et al., 1996) via the β₃ extracellular domain. It is possible that Sdc-1 also engages directly with the integrin, or with one of these other receptors. Like CD47 (Fujimoto et al., 2003; Lindberg et al., 1996), it is observed that the S1ED, when expressed in cells and engaged with ligand, is sufficient to mediate a functional interaction with the β₃ subunit that alters the conformation of the integrin to a high affinity ligand binding state. However, soluble, recombinant S1ED, which is not tethered to the membrane and unable to sense the mechanical force imbued by an immobilized ligand acts as a functional inhibitor of αᵥβ₃. Intriguingly, soluble recombinant CD87 binds to the αᵥβ₃ integrin (Degryse et al., 2001) and competitively inhibits the physical and functional coupling of CD87 to the integrin (Simon et al., 2000; Wei et al., 1996).

Other studies have implicated the syndecan EDs in important protein interactions at cell surfaces. Sdc-1 and Sdc-4 EDs mediate binding interactions with cultured fibroblasts and endothelial cells (McFall and Rapraeger, 1997; McFall and Rapraeger, 1998). Antibodies that target the EDs of Sdc-1 and Sdc-3 block Schwann cell spreading on LN and FN (Carey et al., 1994) and FGF2-dependent proliferation of cultured chondrocytes (Kirsch et al., 2002) respectively. Competition with recombinant ED is effective in disrupting cell spreading and inducing cell cycle arrest in colon carcinoma cells that overexpress Sdc-2 (Park et al., 2002). Finally, polarization of Sdc-1-expressing Raji cells is dependent on the S1ED (McQuade and Rapraeger, 2003). In each of these cases, the exact mechanism of the extracellular core protein interaction remains unknown.

In addition, it has now been demonstrated that there is also as syndecan link for αᵥβ₅ integrin. The inventors show here that B82L cells rely almost solely on the αᵥβ₅ integrin for attachment and spreading on VN and this integrin activity depends on Sdc-1. Cells ligated by Sdc-1 antibody display hyperactivation of the αᵥβ₅ integrin. In addition, expression studies show that the expression of the extracellular domain of Sdc-1 at the cell surface is necessary for integrin activation. In keeping with this finding, competition with the recombinant ectodomain of Sdc-1 inactivates the integrin, as shown by the failure of cells to attach and spread to the αᵥβ₅ integrin ligand VN.

In the B82L fibroblasts, it is envisioned that the syndecan assembles into a cell surface signaling complex that is necessary for αᵥβ₅ integrin signaling, although it is not entirely clear what other receptors, if any, are in the complex. What are the features of this complex? One feature is that anchorage of the syndecan to the substratum appears to lower the threshold for integrin activation by VN or FN. Thus, if Sdc-1 is engaged by antibody, then low concentrations of matrix ligand appear sufficient to activate the integrin and lead to signaling. The simplest explanation would appear to be that the syndecan simply anchors the cell to the substratum so that the integrin can engage the limited amounts of matrix ligand. However, this explanation appears to be ruled out by the fact that engagement of Sdc-4 with antibody, which also anchors the cells to the substratum, does not result in αᵥβ₅ integrin-dependent signaling. Alternatively, it is possible that the specificity arises from Sdc-1 and the αᵥβ₅ integrin being in a complex together such that anchorage of Sdc-1 would cluster the integrin as well as bring it into close apposition to the matrix ligands. As such, cells adhering via Sdc-4 are likely not sensitized/primed to bind VN and FN presumably due to the failure of Sdc-4, which possesses a very different ectodomain both in size and sequence relative to Sdc-1, to interact with the integrin.

A second feature is that the syndecan HS chains are not required for integrin activation, either on syndecan antibody or on matrix ligand. The syndecan HS chains, at least on the B82L cells, do not appear to bind VN or FN sufficiently well for the cells to strongly adhere and cell binding occurs only at sufficiently high matrix concentrations for the integrin to become engaged. Here, the high concentration of matrix ligand is presumably able to bind and activate the integrin and to trigger outside-in signaling. Nonetheless, this signaling also requires Sdc-1 as a third feature of this complex is that the integrin-mediated cell adhesion and spreading on these matrix ligands is blocked by recombinant S1ED and by selective downregulation of Sdc-1 expression by siRNA. Integrin-mediated cell spreading on VN is rescued in mouse Sdc-1-siRNA transfected cells by expression of full-length human Sdc-1 but not expression of a Sdc-1 mutant lacking its ectodomain (FcR^{ecto}-hS1). Moreover, immunoprecipitation of the syndecan brings down β₅ integrin with full-length Sdc-1, either mouse or human, but not with FcR^{ecto}-hS1 despite the fact that this chimera retains the human Sdc-1 transmembrane and cytoplasmic domains. These features suggest that the syndecan and the integrin are in a complex together and that interactions of the Sdc-1 ectodomain within the complex, which can be disrupted by soluble S1ED or by siRNA-mediated removal of the syndecan from the cell surface, are necessary for αᵥβ₅ integrin signaling.

In summary, the inventors have elucidated a mechanism in which the activities and functions of the αᵥβ₃ and αᵥβ₅ integrins are directly modulated by their physical or functional coupling to Sdc-1. As such, Sdc-1 is likely to be a critical regulator of the αᵥβ₃and αᵥβ₅ integrins in the multiple cell behaviors that rely on these integrins. The details of how the invention maybe exploited are described in detail below.

### II. SYNDECANS

A. The Syndecan Family

Cell surface adhesion receptors physically bind cells to their extracellular matrix (ECM) and couple such interactions to intracellular signaling mechanisms which influence gene expression, cell morphology, motility, growth, differentiation and survival (Roskelley et al., 1995; Miranti and Brugge, 2002). Many ECM ligands contain closely spaced proteoglycan- and integrin-binding domains, indicating that the molecular mechanisms by which cells recognize and interact with their extracellular milieu may involve the formation of signaling complexes containing both proteoglycans and integrins. Consequentially, these two types of receptors may act in concert to modulate cell adhesion and migration. While the role of integrins in cell adhesion and signaling is well established, the role of heparan sulfate proteoglycans (HSPGs) is not well characterized.

The vertebrate syndecans are a family of four transmembrane HSPGs. Endowed by their heparan sulfate (HS) chains, syndecans bind a variety of ECM ligands, including fibronectin (FN), laminin (LN), tenascin, thrombospondin (TSP), vitronectin (VN) and the fibrillar collagens (COL) (Bernfield et al., 1999). While the syndecan HS chains are essential for matrix binding, less is known about the role of syndecan core proteins in cell adhesion signaling, although the core protein can affect ligand binding interactions, as well as occupancy induced signaling (Rapraeger and Ott, 1998; Rapraeger, 2000).

The syndecans display a high degree of conservation within their core proteins both across species and across family members. Like the integrins, the syndecans lack intrinsic signaling activity. Their short cytoplasmic tails (ca. 30 aa) consist of three regions, two of which are conserved amongst the four syndecans (C1 and C2) and which flank an intervening variable (V) region. Proteins known to interact with these conserved domains are believed to link syndecan ligand binding interactions to the transduction of intracellular signals (Couchman et al., 2001). Each family member is uniquely defined by its ectodomains and the V-regions of its cytoplasmic tail. Divergence within these regions is believed to confer separate and distinct functions to each individual family member. Distinct roles for the V-regions of Sdc-2 and -4 in matrix assembly and focal adhesion formation respectively have been described (Klass et al., 2000; Woods and Couchman, 2001); however, specific functions for the syndecan ectodomains are almost wholly unknown with the noted exception of Sdc-1 and -4 which contain binding sites for as yet unidentified cell surface receptor(s) (McFall and Rapraeger, 1997; McFall and Rapraeger, 1998).

B. Syndecan Function in Cell Adhesion and Spreading

Current evidence suggests that the syndecan core proteins participate in adhesion-mediated signaling in collaboration with co-receptors at the cell surface. One example is Sdc-4 in focal adhesion and stress fiber formation, which requires both Sdc-4 and integrin engagement whereas neither is sufficient alone (Woods et al., 1986; Izzard et al., 1986; Streeter and Rees,1987; Singer et al., 1987). The requirement for Sdc-4 ligation can be overcome by treatment with phorbol esters (Woods and Couchman, 1994) or lysophosphatidic acid (LPA) (Saoncella et al., 1999) implicating PKC and RhoA in Sdc-4 signaling. While the mechanism by which Sdc-4 contributes to RhoA activation is not clear, it is known that Sdc-4 interacts directly with PKCα as well as phosphatidyl inositol 4,5 bisphosphate (PIP2) via its cytoplasmic tail and these interactions potentiate PKCα activity (Oh et al., 1997a; Oh et al., 1997b; Oh et al., 1998; Baciu and Goetinck, 1995).

While the mechanism by which Sdc-1 signals is not clear, there is ample evidence implicating a signaling role for this receptor as well. Ectopic expression of Sdc-1 in Schwann cells enhances cell spreading and promotes the formation of focal adhesions (Hansen et al., 1994) and actin stress fibers (Carey et al., 1994a); similar morphological changes occur when Sdc-1 is co-clustered with antibodies (Carey et al., 1994b). This response requires the cytoplasmic domain, since clustering of a truncated core protein did not induce reorganization of the cytoskeleton. Expression of Sdc-1 in human ARH-77 leukemia cells or hepatocellular carcinoma cells inhibits invasion of cells into COL matrices (Liu et al., 1998; Ohtake et al., 1999). ARH-77 cells expressing a chimera comprised of the Sdc-1 ectodomain fused to the glycosyl-phosphatidyl inositol (GPI) tail of glypican-1 also fail to invade a COL matrix demonstrating that Sdc-1 's anti-invasive activity resides in its extracellular domain. In similar studies, Raji human lymphoblastoid cells transfected with mouse Sdc-1 (Raji-S1) spread on TSP, FN and antibodies directed against the Sdc-1 ectodomain (Lebakken and Rapraeger, 1996). This spreading is unaffected by truncation of the cytoplasmic domain, indicating that the Sdc-1 core protein interacts with and cooperatively signals through an associated transmembrane signaling partner. Analogous features have also been observed for Sdc-2 (Granes et al., 1999)and Sdc-4 (Yamashita et al., 1999).

Potential syndecan signaling partners include cell surface integrins. Iba et al. (2000) demonstrated that mesenchymal cells when seeded on an HS-specific ligand, the cysteine rich domain of a disintegrin and metalloprotease, ADAM-12/Meltrin α (rADAM12-cys), will spread in a manner that requires cooperate signaling of both syndecans and β₁ integrins. These results imply that syndecan(s) can trigger signaling cascades required for cell spreading either by exposing a cryptic binding site for β₁ integrins, as has been proposed for FN (Khan et al., 1988), or by modulating the activation state of β₁ integrins. Interestingly, colon carcinoma cells attach but fail to spread on aADAM12-cys. However, exogenous stimulation of β₁ integrins with Mn²⁺ or β₁ integrin function activating antibody, mAb 12G10, induced cell spreading, suggesting a mechanism whereby the syndecan activates β₁ integrins is blocked in transformed cells.

C. Angiogenesis

The formation of new blood vessels (called angiogenesis), which occurs in development and disease, relies on inducing proliferation and migration of endothelial cells, and in controlling the survival or apoptosis of the cells to control the architecture of the new vessels (vascular pruning). The αᵥβ3 integrin has important roles in all three of these steps.

FGF and VEGF, two growth factors that are often released by tumors, cause endothelial cells to undergo angiogenesis. Blood vessels in the vicinity of the tumor respond to VEGF by becoming leaky (thus the name vascular permeability factor) allowing fibronectin, vitronectin and fibrinogen in the blood to infiltrate the surrounding matrix. These matrix ligands are critical adhesion and activation ligands for the αᵥβ3 and αᵥβ5 integrins, which have roles in the chemotactic migration of the endothelial cells and in the survival of the cells during vessel pruning. A second response to the growth factors, particularly FGF, is the activation of a neovessel development program that relies on Hox master genes (Boudreau et al., 1997; Myers et al., 2000; 2002). HoxD3 is initially expressed and controls a family of genes that are necessary for the initial migration process, including upregulation of the αᵥβ3 integrin, MMPs and uPAR (Boudreau et al., 1997). Expression of HoxD3 is followed by HoxB3 that regulates the morphogenesis leading to formation of small vessels (Myers et al., 2000), and finally by the HoxD10 gene, which restores the mature phenotype of the cells (Myers et al., 2002); it is HoxD10 that is expressed in resting, stable blood vessels in vivo.

The αᵥβ3 integrin is important not only during endothelial cell migration, but is an important player in the survival of the endothelial cells. Although endothelial cells in mature vessels are not readily susceptible to apoptosis, angiogenic cells that are induced by growth factors rely upon the continued presence of these factors for survival. This is shown experimentally by inducing angiogenesis with VEGF and causing apoptosis by its withdrawal, or in vivo when the developing ovarian follicle induces angiogenesis by release of VEGF and the newly formed bloods vessels regress upon ovulation as the source of VEGF is lost. Endothelial cells responding to VEGF have been shown to be dependent on signaling from the αᵥβ5 integrin in order to block this apoptotic process (Brooks et al., 1994; Friedlander et al., 1995). Thus, inhibiting the integrin using anti-integrin antibodies will induce apoptosis of the endothelial cells responding to VEGF. Similarly, endothelial cells responding to fibroblast growth factor (FGF) are susceptible to apoptosis unless there is coordinate signaling from the αᵥβ5 integrin.

There are a number of anti-angiogenic compounds that have been described and many are in clinical trials. Some are generated in vivo, potentially the proteolysis of native matrix components, giving rise to angiostatin (O'Reilly et al., 1994), endostatin (O'Reilly et al., 1997), canstatin (Kamphaus et al., 2000), arresten (Colorado et al., 2000) and tumstatin (Maeshima et al., 2000). Tumstatin is of interest as it is derived from the α3 chain of Collagen IV and the active site within tumstatin is a peptide binding site that targets the αᵥβ3 integrin, although it appears distinct from the RDG binding site of the integrin. Tumstatin inhibits the proliferation of endothelial cells and is a highly effective inhibitor in angiogenesis assays. As this invention describes the regulation of the αᵥβ3 integrin by Sdc-1 in mammary carcinoma cells and in endothelial cells, and this dependence can be blocked by soluble S1ED, it is hypothesized that this inhibitor would be an effective inhibitor of angiogenesis as well. Perhaps more intriguing is its activity only against the endothelial cells that express Sdc-1. Although there is not much information available, the information to date indicates that resting endothelial cells lining adult blood vessels do not express Sdc-1. In contrast, expression of Sdc-1 is turned on when the cells are activated to undergo angiogenesis, such as occurs normally in wounding, or occurs in abnormal conditions such as diabetic retinopathy, restinosis following blood catheter injury to blood vessels, or tumor angiogenesis. Thus, it is intriguing that targeting the Sdc-1 regulation of the αᵥβ3integrin can provide not only an additional opportunity for drug discovery, but the drug may be most efficacious during angiogenesis itself.

There has not been a concerted examination of Sdc-1 expression in vascular endothelium. Most reports suggest that it is expressed poorly or not at all on resting, mature vascular endothelium that lines blood vessels. However, there are reports that suggest it is expressed on activated endothelial cells participating in angiogenesis in the wounded skin (Elenius et al., 1991; Gallo et al., 1996). Sdc-1 is not expressed in endothelial cells lining the rabbit aorta, but expression is upregulated following balloon catheter injury and persists for up to 12 weeks following injury. There is a report that Sdc-1 is upregulated in a subset of vessels during tumor angiogenesis (Gotte et al., 2002). These studies strongly suggest that Sdc-1 becomes expressed on activated cells responding to injury or growth factors. Cultured cells, such as human aortic and human umbilical vein endothelial cells show expression at both the protein and mRNA level, although expression in human umbilical vein endothelial cells is low (Mertens et al., 1992). However, the expression patterns described may be dependent on the growth factors and supplements, such as brain extract, added to the culture medium.

D. Syndecan-1

Syndecan-1 is highly expressed at the basolateral surface of epithelial cells where it is thought to interact with the actin cytoskeleton and to modulate cell adhesion and growth factor signaling (Bernfield et al., 1999; Rapraeger et al., 1986; Kim et al., 1994; Sanderson and Bernfield, 1988). In experimental studies of malignant transformation, Sdc-1 expression is associated with the maintenance of epithelial morphology, anchorage-dependent growth and inhibition of invasiveness. Alterations in syndecan expression during development (Sun et al., 1998) and in transformed epithelial (Inki and Jalkanen, 1996; Bayer-Garner et al., 2001) are associated with an epithelial-mesenchymal transformation with attendant alterations in cell morphology, motility, growth and differentiation. Transfection of epithelial cells with anti-sense mRNA for Sdc-1 or downregulation of Sdc-1 expression by androgeninduced transformation results in an epithelial to mesenchymal transformation and increased invasion (Leppa et al., 1992; Kato et al., 1995; Leppa et al., 1991). The loss of E-cadherin under these circumstances has long suggested a coordinate regulation of Sdc-1 and E-cadherin expression (Sun et al., 1998; Leppa et al., 1996). These studies, as well as others, indicate that there appears to be a threshold requirement for syndecan expression to elicit its biological activity. Syndecan-1 is downregulated in a number of epithelial cancers and in pre-malignant lesions of the oral mucosa (Soukka et al., 2000) and uterine cervix (Inki et al., 1994; Rintala et al., 1999; Nakanishi et al., 1999), and its loss may be an early genetic event contributing to tumor progression (Sanderson, 2001;Numa et al., 2002; Hirabayashi et al., 1998). Loss of Sdc-1 correlates with a reduced survival in squamous cell carcinoma of the head, neck and lung (Anttonen et al., 1999; Inki et al., 1994; Nakaerts et al., 1997), laryngeal cancer (Pulkkinen et al., 1997; Klatka, 2002), malignant mesothelioma (Kumar-Singh et al., 1998) and multiple myeloma (Sanderson and Borset, 2002) and a high metastatic potential in hepatocellular and colorectal carcinomas (Matsumoto et al., 1997; Fujiya et al., 2001; Levy et al., 1997; Levy et al., 1996). Downregulation of Sdc-2 and -4 expression has also been observed in certain human carcinomas (Nakaerts et al., 1997; Park et al., 2002; Mundhenke et al., 2002; Crescimanno et al., 1999), but the functional consequences of these alterations in expression are less clear.

In contrast to the general notion that the syndecan may be an inhibitor of carcinogenesis, Sdc-1 also demonstrates tumor promoter function. Syndecan-1 supplements Wnt-1 induced tumorigenesis of the mouse mammary gland (Alexander et al., 2000) and promotes the formation of metastases in mouse lung squamous carcinoma cells (Hirabayashi et al., 1998). Enhanced Sdc-1 expression has also been observed in pancreatic (Conejo et al., 2000), gastric (Wiksten et al., 2001) and breast (Burbach et al., 2003; Stanley et al., 1999; Barbareschi et al., 2003) carcinomas and this overexpression correlates with increased tumor aggressiveness and poor clinical prognosis. This duality in the role of Sdc-1 in tumorigenesis may reflect tissue and/or tumor stage-specific function, or reflect the multiple functions of this PG.

Sanderson was the first to demonstrate a role for Sdc-1 in tumor cell migration by examining the invasion of myeloma cells into collagen gels (Liu et al., 1998). Ectopic expression of Sdc-1 in syndecan-deficient myeloma cells had the striking effect of curtailing invasion, whereas the expression of other cell surface heparan sulfate PGs (e.g., glypican) was without effect. Using chimeras derived from these two proteins, Sanderson showed that the activity of the syndecan is preserved when its ectodomain alone is expressed as a glycosyl-phosphatidylinositol (GPI)-linked protein at the cell surface. Although clearly responsible for binding the collagen matrix via its attached heparan sulfate chains, the anti-invasive activity of the syndecan requires yet an additional interaction that traces to a site in the extracellular domain of the core protein itself. The mechanism by which the ectodomain site influences the invasion of the myeloma cells is unknown, but its interaction with other cell surface receptors in a “co-receptor” role is one possibility. More recently, ectopic expression of Sdc-1 has also been shown to curtail the invasion of hepatocellular carcinoma cells into a collagen matrix (Ohtake et al., 1999).

E. Proteins and Peptides

Syndecan-1 peptides and polypeptides of the present invention will generally comprise molecules of 5 to about 240 residues in length, and may have the sequence SEQ ID NO:4 or SEQ ID NO:8 or SEQ ID NO:9. A particular length may be 234 residues, 34 residues, less than 30 residues, less than 25 residues, less than 20 residues, less than 15 residues, or less than 14 residues, including 5, 6, 7, 8, 9, 10, 11, 12, or 13 residues. In other embodiments, the peptides or proteins may be from SEQ ID NOS:2, 4, 8 or 9, and may thus comprise 40, 50, 60, 70, 80, 90, 100, 110, 120, 130, 140, 150, 160, 170, 180, 190, 200, 210, 220, 230, or 240 consecutive residues of that sequence. The peptides or proteins may be generated synthetically or by recombinant techniques, and are purified according to known methods, such as precipitation (e.g., ammonium sulfate), HPLC, ion exchange chromatography, affinity chromatography (including immunoaffinity chromatography) or various size separations (sedimentation, gel electrophoresis, gel filtration).

Accordingly, sequences that have between about 70% and about 80%, between about 81% and about 90%, between about 91% and 95%, or about 96, about 97%, 98% or about 99% of amino acids that are identical or functionally equivalent to the amino acids of SEQ ID NOS:2, 4, 8 or 9, will be sequences that are “essentially as set forth in SEQ ID NOS:2, 4, 8 or 9.”

i. Substitutional Variants

It also is contemplated in the present invention that variants or analogs of syndecan-1 peptides or proteins may also inhibit tumor growth. Polypeptide sequence variants of syndecan-1, primarily making conservative amino acid substitutions to SEQ ID NOS:2, 4, 8 or 9, may provide improved compositions. Substitutional variants typically contain the exchange of one amino acid for another at one or more sites within the protein, and may be designed to modulate one or more properties of the polypeptide, such as stability against proteolytic cleavage, without the loss of other functions or properties. Substitutions of this kind preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

The following is a discussion based upon changing of the amino acids of a peptide or protein to create an equivalent, or even an improved, second-generation molecule. For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a peptide that defines that peptide's biological functional activity, certain amino acid substitutions can be made in a protein sequence, and its underlying DNA coding sequence, and nevertheless obtain a peptide with like properties. It is thus contemplated by the inventors that various changes may be made in syndecan-1 amino acid sequences and in the DNA sequences coding the peptide without appreciable loss of their biological utility or activity, as discussed below.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant peptide, which in turn defines the interaction of the peptide with other molecules.

Each amino acid has been assigned a hydropathic index on the basis of their hydrophobicity and charge characteristics (Kyte and Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a peptide with similar biological activity, i.e., still obtain a biological functionally equivalent protein. In making such changes, the substitution of amino acids whose hydropathic indices are within ±2 is preferred, those which are within +1 are particularly preferred, and those within +0.5 are even more particularly preferred.

It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine *−0.5); cysteine (−1.0); methionine (−1.3); valine (−1.5); leucine (−1.8); isoleucine (−1.8); tyrosine (−2.3); phenylalanine (−2.5); tryptophan (−3.4).

It is understood that an amino acid can be substituted for another having a similar hydrophilicity value and still obtain a biologically equivalent and immunologically equivalent protein. In such changes, the substitution of amino acids whose hydrophilicity values are within ±2 is preferred, those that are within +1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine.

Another embodiment for the preparation of polypeptides according to the invention is the use of peptide mimetics. Mimetics are peptide containing molecules that mimic elements of protein secondary structure (Johnson et al, 1993). The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles may be used, in conjunction with the principles outline above, to engineer second generation molecules having many of the natural properties of syndecan-1, but with altered and even improved characteristics.

ii. Altered Amino Acids

The present invention may employ peptides that comprise modified, non-natural and/or unusual amino acids. A table of exemplary, but not limiting, modified, non-natural and/or unusual amino acids is provided herein below. Chemical synthesis may be employed to incorporated such amino acids into the peptides of interest.

TABLE 1
Modified, Non-Natural and Unusual Amino Acids

| Abbr. | Amino Acid |
|---|---|
| Aad | 2-Aminoadipic acid |
| BAad | 3-Aminoadipic acid |
| BAla | beta-alanine, beta-Amino-propionic acid |
| Abu | 2-Aminobutyric acid |
| 4Abu | 4-Aminobutyric acid, piperidinic acid |
| Acp | 6-Aminocaproic acid |
| Ahe | 2-Aminoheptanoic acid |
| Aib | 2-Aminoisobutyric acid |
| BAib | 3-Aminoisobutyric acid |
| Apm | 2-Aminopimelic acid |
| Dbu | 2,4-Diaminobutyric acid |
| Des | Desmosine |
| Dpm | 2,2′-Diaminopimelic acid |
| Dpr | 2,3-Diaminopropionic acid |
| EtGly | N-Ethylglycine |
| EtAsn | N-Ethylasparagine |
| Hyl | Hydroxylysine |
| Ahyl | allo-Hydroxylysine |
| 3Hyp | 3-Hydroxyproline |
| 4Hyp | 4-Hydroxyproline |
| Ide | Isodesmosine |
| Aile | allo-Isoleucine |
| MeGly | N-Methylglycine, sarcosine |
| MeIle | N-Methylisoleucine |
| MeLys | 6-N-Methyllysine |
| MeVal | N-Methylvaline |
| Nva | Norvaline |
| Nle | Norleucine |
| Orn | Ornithine |

iii. Mimetics

In addition to the variants discussed above, the present inventors also contemplate that structurally similar compounds may be formulated to mimic the key portions of peptide or polypeptides of the present invention. Such compounds, which may be termed peptidomimetics, may be used in the same manner as the peptides of the invention and, hence, also are functional equivalents.

Certain mimetics that mimic elements of protein secondary and tertiary structure are described in Johnson et al. (1993). The underlying rationale behind the use of peptide mimetics is that the peptide backbone of proteins exists chiefly to orient amino acid side chains in such a way as to facilitate molecular interactions, such as those of antibody and/or antigen. A peptide mimetic is thus designed to permit molecular interactions similar to the natural molecule.

Some successful applications of the peptide mimetic concept have focused on mimetics of P-turns within proteins, which are known to be highly antigenic. Likely β-turn structure within a polypeptide can be predicted by computer-based algorithms, as discussed herein. Once the component amino acids of the turn are determined, mimetics can be constructed to achieve a similar spatial orientation of the essential elements of the amino acid side chains.

Other approaches have focused on the use of small, multidisulfide-containing proteins as attractive structural templates for producing biologically active conformations that mimic the binding sites of large proteins (Vita et al., 1998). A structural motif that appears to be evolutionarily conserved in certain toxins is small (30-40 amino acids), stable, and high permissive for mutation. This motif is composed of a beta sheet and an alpha helix bridged in the interior core by three disulfides. Beta II turns have been mimicked successfully using cyclic L-pentapeptides and those with D-amino acids (Weisshoff et al., 1999). Also, Johannesson et al. (1999) report on bicyclic tripeptides with reverse turn inducing properties.

Methods for generating specific structures have been disclosed in the art. For example, alpha-helix mimetics are disclosed in U.S. Pat. Nos. 5,446,128; 5,710,245; 5,840,833; and 5,859,184. These structures, which render the peptide or protein more thermally stable, also increase resistance to proteolytic degradation. Six, seven, eleven, twelve, thirteen and fourteen membered ring structures are disclosed.

Methods for generating conformationally restricted beta turns and beta bulges are described, for example, in U.S. Pat. Nos. 5,440,013; 5,618,914; and 5,670,155. Beta-turns permit changed side substituents without having changes in corresponding backbone conformation, and have appropriate termini for incorporation into peptides by standard synthesis procedures. Other types of mimetic turns include reverse and gamma turns. Reverse turn mimetics are disclosed in U.S. Pat. Nos. 5,475,085 and 5,929,237, and gamma turn mimetics are described in U.S. Pat. Nos. 5,672,681 and 5,674,976.

iv. D Amino Acids

In another form, the present invention contemplates use of variants that comprise various portions of an syndecan-1 peptide or protein in reverse order of SEQ ID NOS:2, 4, 8 or 9, using D amino acids, stereoisomers of natural amino acids which are in the L-form.

### III. PEPTIDE SYNTHESIS

Syndecan-1 and related peptides may be generated synthetically for use in various embodiments of the present invention. Because of their relatively small size, the peptides of the invention can be synthesized in solution or on a solid support in accordance with conventional techniques. Various automatic synthesizers are commercially available and can be used in accordance with known protocols. See, for example, Stewart and Young (1984); Tam et al. (1983); Merrifield (1986); Barany and Merrifield (1979), each incorporated herein by reference. Short peptide sequences, or libraries of overlapping peptides, usually from about 5 up to about 34 to 40 amino acids, which correspond to the selected regions described herein, can be readily synthesized and then screened in screening assays designed to identify reactive peptides. Alternatively, recombinant DNA technology may be employed wherein a nucleotide sequence which encodes a peptide of the invention is inserted into an expression vector, transformed or transfected into an appropriate host cell and cultivated under conditions suitable for expression.

It may be desirable to purify syndecan-1 variants, peptide-mimics or analogs thereof. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. A particularly efficient method of purifying peptides is fast protein liquid chromatography or even HPLC.

Certain aspects of the present invention concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand. One of the most common forms of affinity chromatography is immunoaffinity chromatography. The generation of antibodies that would be suitable for use in accord with the present invention is discussed below.

### IV. ANTI-IDIOTYPIC ANTIBODIES

The present invention also provide antibodies that mimic the syndecan-1 peptides and proteins described herein. These antibodies are created by first preparing an antibody against a syndecan-1 peptide or protein and then preparing a second antibody, called an anti-idiotypic antibody, against the idiotype of the first antibody. As a mirror image of a mirror image, the binding site of anti-idiotype would be expected to be an analog of the original antigen. As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

The term “antibody” is used to refer to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibodies and antibody-based constructs and fragments are well known in the art (see, e.g., Harlow et al., 1988; and U.S. Pat. No. 4,196,265 each incorporated herein by reference).

### V. NUCLEIC ACID SEGMENTS ENCODING SYNDECAN PEPTIDES AND PROTEINS

The present invention concerns nucleic acid segments, isolatable from cells, that are free from total genomic DNA and that are capable of expressing all or part of a protein or polypeptide such as syndecan-1 or the syndecan-1 peptide or protein of SEQ ID NOS:2, 4, 8 or 9. The nucleic acid may encode a peptide or polypeptide containing all or part of the syndecan-1 amino acid sequence.

As used herein, the term “nucleic acid segment” refers to a DNA molecule that has been isolated free of total genomic DNA of a particular species. Therefore, a nucleic acid segment encoding a syndecan-1 refers to a nucleic acid segment that contains wild-type, polymorphic, or mutant polypeptide-coding sequences yet is isolated away from, or purified free from, total mammalian or human genomic DNA. Included within the term “nucleic acid segment” are a polypeptide(s), nucleic acid segments smaller than a polypeptide, and recombinant vectors, including, for example, plasmids, cosmids, phage, viruses, and the like.

The term “cDNA” is intended to refer to DNA prepared using messenger RNA (mRNA) as template. The advantage of using a cDNA, as opposed to genomic DNA or DNA polymerized from a genomic, non- or partially-processed RNA template, is that the cDNA primarily contains coding sequences of the corresponding protein. There may be times when the full or partial genomic sequence is preferred, such as where the non-coding regions are required for optimal expression or where non-coding regions such as introns are to be targeted in an antisense strategy.

A nucleic acid segment encoding all or part of a native or modified polypeptide may contain a contiguous nucleic acid sequence encoding all or a portion of such a polypeptide of the following lengths: about 10, about 20, about 30, about 40, about 50, about 60, about 70, about 80, about 90, or about 100, about 110, about 120, about 130, about 140, about 150, about 160, about 170, about 180, about 190, about 200, about 210, about 220, about 230, about 240, about 250 or about 260 nucleotides, nucleosides, or base pairs. Specifically contemplate are a segment encoding SEQ ID NO:2, the 34 residue peptide of SEQ ID NO:4, a segment encoding residues 18-251 residues of SEQ ID NO:2 (SEQ ID NO:8) or residues 18-310 of SEQ ID NO:2 (SED ID NO:9).

The nucleic acid segments used in the present invention, regardless of the length of the coding sequence itself, may be combined with other nucleic acid sequences, such as promoters, enhancers polyadenylation signals, origin of replication, and a selectable marker gene, as well as other coding segments, and the like (all as are known to those of ordinary skill in the art), such that their overall length may vary considerably.

The term oligonucleotide refers to at least one molecule of between about 3 and about 100 nucleobases in length. These definitions generally refer to at least one single-stranded molecule, but in specific embodiments will also encompass at least one additional strand that is partially, substantially or fully complementary to the at least one single-stranded molecule. Thus, a nucleic acid may encompass at least one double-stranded molecule or at least one triple-stranded molecule that comprises one or more complementary strand(s) or “complement(s)” of a particular sequence comprising a strand of the molecule.

It is contemplated that the nucleic acid constructs of the present invention may encode full-length polypeptide from any source or encode a truncated version of the polypeptide, for example a truncated syndecan-1 polypeptide, such that the transcript of the coding region represents the truncated version. The truncated transcript may then be translated into a truncated protein. Alternatively, a nucleic acid sequence may encode a full-length polypeptide sequence with additional heterologous coding sequences, for example to allow for purification of the polypeptide, transport, secretion, post-translational modification, or for therapeutic benefits such as targeting or efficacy.

It is contemplated that the nucleic acid constructs of the present invention may regulate gene expression of an immunogenic polypeptide. A nucleic acid segment may regulate the expression of a full-length polypeptide sequence with additional heterologous coding sequences, for example to allow for therapeutic benefits such as targeting or efficacy.

In a non-limiting example, one or more nucleic acid constructs may be prepared that include a contiguous stretch of nucleotides identical to or complementary to a particular gene, such as the human syndecan-1 gene (SEQ ID NO:1), or a fragment thereof (SEQ ID NO:3 or that encoding SEQ ID NOS:8 or 9). Such a nucleic acid construct may be at least 20, 30, 40, 50, 60, 70, 80, 90, 100, 110, 120, 130, 140, 150, 160, 170, 180, 190, 200, 250, 300, 400, 500, 600, 700, 800, 900, 1,000, 2,000, 3,000, 4,000, 5,000, 6,000, 7,000, 8,000, 9,000, 10,000, 15,000, 20,000, 30,000, 50,000, 100,000, 250,000, 500,000, 750,000, to at least 1,000,000 nucleotides in length, as well as constructs of greater size, up to and including chromosomal sizes (including all intermediate lengths and intermediate ranges), given the advent of nucleic acids constructs such as a yeast artificial chromosome are known to those of ordinary skill in the art. It will be readily understood that “intermediate lengths” and “intermediate ranges,” as used herein, means any length or range including or between the quoted values (i.e., all integers including and between such values).

In certain other embodiments, the invention concerns isolated nucleic acid segments and recombinant vectors that include within their sequence a contiguous nucleic acid sequence from that shown in SEQ ID NO: 1, 3 or encoding SEQ ID NOS: 8 or 9. This definition is used in the same sense as described above and means that the nucleic acid sequence substantially corresponds to a contiguous portion of that shown in SEQ ID NO:1, 3 or encoding SEQ ID NOS: 8 or 9 and has relatively few codons that are not identical, or functionally equivalent, to the codons of SEQ ID NO:1, 3 or encoding SEQ ID NOS: 8 or 9. The term “functionally equivalent codon” is used herein to refer to codons that encode the same amino acid, such as the six codons for arginine or serine, and also refers to codons that encode biologically equivalent amino acids, as is known to those of skill in the art.

It also will be understood that this invention is not limited to the particular nucleic acid sequence of SEQ ID NO:1 and the amino acid sequence of SEQ ID NO:2. Recombinant vectors and isolated DNA segments may therefore variously include the syndecan-1-coding regions themselves, coding regions bearing selected alterations or modifications in the basic coding region, or they may encode larger polypeptides that nevertheless include syndecan-1-coding regions or may encode biologically functional equivalent proteins or peptides that have variant amino acids sequences.

The nucleic acid segments of the present invention encompass biologically functional equivalent syndecan-1 proteins and peptides. Such sequences may arise as a consequence of codon redundancy and functional equivalency that are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by man may be introduced through the application of site-directed mutagenesis techniques, e.g., to introduce improvements to the antigenicity of the protein.

If desired, one also may prepare fusion proteins and peptides, e.g., where the syndecan-1-coding regions are aligned within the same expression unit with other proteins or peptides having desired functions, such as for purification or immunodetection purposes (e.g., proteins that may be purified by affinity chromatography and enzyme label coding regions, respectively).

Encompassed by certain embodiments of the present invention are nucleic acid segments encoding relatively small peptides, such as, for example, peptides of from about 5 to about 40 amino acids in length, and more preferably, of from about 10 to about 34 amino acids in length; and also larger polypeptides up to and including proteins corresponding to the full-length sequence set forth in SEQ ID NO:2, the peptide of SEQ ID NO:4 or the polypeptide of SEQ ID NOS:8 or 9, or to specific nucleic acid fragments of SEQ ID NO:1, such as SEQ ID NO:3 and those encoding SEQ ID NOS:8 or 9.

A. Promoters

The present invention may also involve expression of sdc-1 or related peptide from a sdc-1-encoding nucleic acid. This requires the presence of a promoter operably linked to the sdc-1-coding region. A promoter generally comprises a nucleic acid sequence that functions to position the start site for RNA synthesis. A promoter may or may not be used in conjunction with an enhancer, which refers to a cis-acting regulatory sequence involved in the transcriptional activation of a nucleic acid sequence. In the present invention, a nucleic acid encoding a sdc-1 comprises a promoter such as a tissue specific promoter, or a constitutive promoter, or an inducible promoter.

A promoter in the context of the present invention may be one naturally associated with a gene or sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter or enhancer, which refers to a promoter or enhancer, that is not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. No. 4,683,202, U.S. Pat. No. 5,928,906, each incorporated herein by reference). Furthermore, it is contemplated the control sequences that direct transcription and/or expression of sequences within non-nuclear organelles such as mitochondria, chloroplasts, and the like, can be employed as well.

Those of skill in the art of molecular biology generally know the use of promoters, enhancers, and cell type combinations for protein expression, for example, see Sambrook et al. (2000), incorporated herein by reference.

The present invention also contemplates the use of tissue specific promoters and inducible promoters. Other promoters that may be employed with the present invention are constitutive and inducible promoters as are well known to those of skill in the art. Additionally any promoter/enhancer combination (as per the Eukaryotic Promoter Data Base EPDB) could also be used to drive expression of structural genes encoding oligosaccharide processing enzymes, protein folding accessory proteins, selectable marker proteins or a heterologous protein of interest.

B. Origins of Replication/Polyadenylation Signals

In expression, particularly eukaryotic expression, one will typically include a polyadenylation signal to effect proper polyadenylation of the transcript. The nature of the polyadenylation signal is not believed to be crucial to the successful practice of the invention. Polyadenylation signals include the SV40 polyadenylation signal and the bovine growth hormone polyadenylation signal, known to function well in various target cells. Polyadenylation may increase the stability of the transcript or may facilitate cytoplasmic transport.

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

C. Delivery of Nucleic Acids

Two broad approaches have been used to employ vectors to deliver nucleic acids to cells, namely viral vectors and non-viral vectors. As by methods described herein and as is known to the skilled artisan, expression vectors may be constructed to deliver nucleic acids segments encoding a syndecan-1 of the present invention to a organelle, cell, tissue, or a subject. Such vectors comprising a syndecan-1 may be used in a variety of manner consistent with the invention, including in screening assay and genetic immunization protocols.

A vector in the context of the present invention refers to a carrier nucleic acid molecule into which a nucleic acid sequence of the present invention may be inserted for introduction into a cell and thereby replicated. A nucleic acid sequence can be exogenous, which means that it is foreign to the cell into which the vector is being introduced; or that the sequence is homologous to a sequence in the cell but positioned within the host cell nucleic acid in which the sequence is ordinarily not found. Vectors include plasmids; cosmids; viruses such as bacteriophage, animal viruses, and plant viruses; and artificial chromosomes (e.g., YACs); and synthetic vectors. One of ordinary skill in the art would be well equipped to construct any number of vectors through standard recombinant techniques as described in Maniatis et al., 1990 and Ausubel et al., 1994, incorporated herein by reference.

Viral vectors may be derived from viruses known to those of skill in the art, for example, bacteriophage, animal and plant virus, including but not limited to, adenovirus, vaccinia virus (Ridgeway, 1988; Baichwal and Sugden, 1986; Coupar et al., 1988) adeno-associated virus (AAV) (Ridgeway, 1988; Baichwal and Sugden, 1986; Hermonat and Muzycska, 1984) retrovirus and herpesvirus and offer several features for use in gene transfer into various mammalian cells (Friedmann, 1989; Ridgeway, 1988; Baichwal and Sugden, 1986; Coupar et al., 1988; Horwich et al., 1990). One of skill in the art would be well equipped to construct a vector through standard recombinant techniques as described in Sambrook et al. (2001), Maniatis et al. (1990) and Ausubel et al. (1994), incorporated herein by reference. The present invention may also employ non-viral vectors.

An expression vector refers to any type of genetic construct comprising a nucleic acid coding for a RNA capable of being transcribed. In the context of the present specification, expression vectors will typically comprise a nucleic acid segment encoding a syndecan-1 as described herein. In some cases, RNA molecules are then translated into a protein, polypeptide, or peptide. In other cases, these sequences are not translated, as in the case of antisense molecules or ribozymes production. Expression vectors can contain a variety of control sequences, which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well, and are described herein

Non-viral vectors, such as plasmids and cosmids, require suitable method for delivery into cells. Such methods include, but are not limited to direct delivery of DNA by: injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), microinjection (Harland and Weintraub, 1985; U. S. Pat. No. 5,789,215, incorporated herein by reference); electroporation (U.S. Pat. No. 5,384,253, incorporated herein by reference; Tur-Kaspa et al., 1986; Potter et al., 1984); calcium phosphate precipitation (Graham and Van Der Eb, 1973; Chen and Okayama, 1987; Rippe et al., 1990); using DEAE-dextran followed by polyethylene glycol (Gopal, 1985); direct sonic loading (Fechheimer et al., 1987); by liposome-mediated transfection (Nicolau and Sene, 1982; Fraley et al., 1979; Nicolau et al., 1987; Wong et al., 1980; Kaneda et al., 1989; Kato et al., 1991); receptor-mediated transfection (Wu and Wu, 1987; Wu and Wu, 1988); microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); agitation with silicon carbide fibers (Kaeppler et al., 1990; U. S. Pat. Nos. 5,302,523 and 5,464,765, each incorporated herein by reference); desiccation/inhibition-mediated DNA uptake (Potrykus et al., 1985), and any combination of such methods. Through the application of techniques such as these, organelle(s), cell(s), tissue(s) or organism(s) may be stably or transiently transformed. In certain embodiments, acceleration methods are preferred and include, for example, microprojectile bombardment or inhalation methods.

Also in context of the present invention, topical delivery of a nucleic acid segment encoding a syndecan-1 to the skin may further comprise vesicles such as liposomes, niosomes and transferosomes thereby enhancing topical and transdermal delivery. Cationic lipids may also be used to deliver negatively charged nucleic acids. Sonophoresis or phonophoresis which involves the use of ultrasound to deliver the nucleic acid of interest, may also be employed for transdermal delivery. Ionotophoresis which consists of applying a low electric field for a period of time to the skin may also be applied in delivering the nucleic acid of interest to the skin.

### VI. PHARMACEUTICAL FORMULATIONS, DELIVERY, AND CANCER TREATMENT REGIMENS

In particular embodiments of the present invention, a method of treatment for cancer by the delivery of a sdc-1 peptide or polypeptide (as described elsewhere in this document) is contemplated. Cancers contemplated by the present invention include, but are not limited to, breast cancer, lung cancer, head and neck cancer, bladder cancer, bone cancer, bone marrow cancer, brain cancer, colon cancer, esophageal cancer, gastrointestinal cancer, gum cancer, kidney cancer, liver cancer, nasopharynx cancer, ovarian cancer, prostate cancer, skin cancer, stomach cancer, testis cancer, tongue cancer, or uterine cancer. In particular embodiments, carcinomas, myelomas, melanomas or gliomas may be treated.

A. Administration

To inhibit a hyperproliferative disease such as cancer, one would generally contact a cell with a sdc-1 peptide or protein or an expression construct encoding a sdc-1 peptide or protein. The preferred method for the delivery of a peptide or an expression construct is via injection. Administration may be parenteral, intradermal, intramuscular, or intratumioral administration. Other administration routes include lavage, continuous perfusion, topical and oral administration and formulation. See U.S. Pat. Nos. 5,543,158; 5,641,515; 5,399,363 (each specifically incorporated herein by reference in its entirety). Injection of nucleic acid constructs of the present invention may be delivered by syringe or any other method used for injection of a solution, as long as the expression construct can pass through the particular gauge of needle required for injection. A novel needleless injection system (U.S. Pat. No. 5,846,233); or a syringe system for use in gene therapy (U.S. Pat. No. 5,846,225), all as incorporated herein by reference, may be employed in the present invention.

B. Compositions and Formulations

Pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. Composition(s) of absorption delay agents(aluminum monostearate and gelatin) may also be used. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

For parenteral administration in an aqueous solution, for example, the solution should be suitably buffered if necessary and the liquid diluent first rendered isotonic with sufficient saline or glucose (see for example, “Remington's Pharmaceutical Sciences” 15th Edition, pages 1035-1038 and 1570-1580). These particular aqueous solutions are especially suitable for subcutaneous, intramuscular, and intratumoral administration. In this connection, sterile aqueous media that may be employed will be known to those of skill in the art in light of the present disclosure. Variation in dosage will necessarily occur depending on the condition of the subject being treated; the severity of the condition, and will be determined by the person administering the dose. For human administration, preparations should meet sterility, pyrogenicity, general safety and purity standards as required by FDA Office of Biologics standards.

The compositions of the present invention may be formulated in a neutral or salt form. Pharmaceutically-acceptable salts, include the acid addition salts (formed with the free amino groups of the protein) and which are formed with inorganic acids; or salts (formed with the free carboxyl groups) derived from inorganic bases as is known to those of ordinary skill in the art.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” or “pharmacologically-acceptable” refers to molecular entities and compositions that do not produce an allergic or similar untoward reaction when administered to a human. The preparation of an aqueous composition that contains a protein as an active ingredient is well understood in the art.

C. Combination Treatments

In the context of the present invention, it is contemplated that sdc-1 peptides, proteins or analogs thereof may be used in combination with an additional therapeutic agent to more effectively treat a cancer.

Additional therapeutic agents contemplated for use in combination with sdc-1 peptides, proteins or analogs thereof include traditional anticancer therapies. Anticancer agents may include but are not limited to, radiotherapy, chemotherapy, gene therapy, hormonal therapy or immunotherapy that targets cancer/tumor cells.

To kill cells, induce cell-cycle arrest, inhibit migration, inhibit metastasis, inhibit survival, inhibit proliferation, or otherwise reverse or reduce the malignant phenotype of cancer cells, using the methods and compositions of the present invention, one would generally contact a cell with sdc-1 peptides, proteins or an analog thereof in combination with an additional therapeutic agent. These compositions would be provided in a combined amount effective to inhibit cell growth and/or induce apoptosis in the cell. This process may involve contacting the cells with sdc-1 peptides, proteins or analogs thereof in combination with an additional therapeutic agent or factor(s) at the same time. This may be achieved by contacting the cell with a single composition or pharmacological formulation that includes both agents, or by contacting the cell with two distinct compositions or formulations, at the same time, wherein one composition includes the sdc-1 peptides, proteins or derivatives thereof and the other includes the additional agent.

Alternatively, treatment with sdc-1 peptides, proteins or analogs thereof may precede or follow the additional agent treatment by intervals ranging from minutes to weeks. In embodiments where the additional agent is applied separately to the cell, one would generally ensure that a significant period of time did not expire between the time of each delivery, such that the agent would still be able to exert an advantageously combined effect on the cell. In such instances, it is contemplated that one would contact the cell with both modalities within about 12-24 hr of each other and, more preferably, within about 6-12 hr of each other, with a delay time of only about 12 hr being most preferred. Thus, therapeutic levels of the drugs will be maintained. In some situations, it may be desirable to extend the time period for treatment significantly (for example, to reduce toxicity). Thus, several days (2, 3, 4, 5, 6 or 7) to several weeks (1, 2, 3, 4, 5, 6, 7 or 8) may lapse between the respective administrations.

It also is conceivable that more than one administration of either syndecan-1 peptides or analogs thereof in combination with an additional anticancer agent will be desired. Various combinations may be employed, where sdc-I peptide, protein or an analog thereof is “A” and the additional therapeutic agent is “B”, as exemplified below:

A/B/A B/A/B B/B/A A/A/B B/A/A A/B/B B/B/B/A
B/B/A/B A/A/B/B A/B/A/B A/B/B/A B/B/A/A B/A/B/A
B/A/A/B B/B/B/A A/A/A/B B/A/A/A A/B/A/A A/A/B/A
A/B/B/B B/A/B/B B/B/A/B

Other combinations are contemplated. Again, to achieve cell killing by the induction of apoptosis, both agents may be delivered to a cell in a combined amount effective to kill the cell.

i. Chemotherapeutic Agents

The present invention also contemplates the use of chemotherapeutic agents in combination with sdc-1 peptides, proteins or an analog thereof in the treatment of cancer. Examples of such chemotherapeutic agents may include, but are not limited to, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, gemcitabien, navelbine, farnesyl-protein tansferase inhibitors, transplatinum, 5-fluorouracil and methotrexate, or any analog or derivative variant of the foregoing.

ii. Radiotherapeutic Agents

Radiotherapeutic agents may also be use in combination with the sdc-1 compounds of the present invention in treating a cancer. Such factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

iii. Immunotherapeutic Agents

Immunotherapeutics may also be employed in the present invention in combination with sdc-1 peptides, proteins or analogs thereof in treating cancer. Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Generally, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

iv. Gene Therapy

The tumor suppressor oncogenes function to inhibit excessive cellular proliferation. The inactivation of these genes destroys their inhibitory activity, resulting in unregulated proliferation. The tumor suppressors p53, p16 and C-CAM are described below.

High levels of mutant p53 have been found in many cells transformed by chemical carcinogenesis, ultraviolet radiation, and several viruses. The p53 gene is a frequent target of mutational inactivation in a wide variety of human tumors and is already documented to be the most frequently mutated gene in common human cancers. It is mutated in over 50% of human NSCLC (Hollstein et al., 1991) and in a wide spectrum of other tumors.

The p53 gene encodes a 393-amino acid phosphoprotein that can form complexes with host proteins such as large-T antigen and E1B. The protein is found in normal tissues and cells, but at concentrations which are minute by comparison with transformed cells or tumor tissue

Wild-type p53 is recognized as an important growth regulator in many cell types. Missense mutations are common for the p53 gene and are essential for the transforming ability of the oncogene. A single genetic change prompted by point mutations can create carcinogenic p53. Unlike other oncogenes, however, p53 point mutations are known to occur in at least 30 distinct codons, often creating dominant alleles that produce shifts in cell phenotype without a reduction to homozygosity. Additionally, many of these dominant negative alleles appear to be tolerated in the organism and passed on in the germ line. Various mutant alleles appear to range from minimally dysfunctional to strongly penetrant, dominant negative alleles (Weinberg, 1991).

Another inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the G₁. The activity of this enzyme may be to phosphorylate Rb at late G₁. The activity of CDK4 is controlled by an activating subunit, D-type cyclin, and by an inhibitory subunit, the p16^{INK4} has been biochemically characterized as a protein that specifically binds to and inhibits CDK4, and thus may regulate Rb phosphorylation (Serrano et al., 1993; Serrano et al., 1995). Since the p16^{INK4} protein is a CDK4 inhibitor (Serrano, 1993), deletion of this gene may increase the activity of CDK4, resulting in hyperphosphorylation of the Rb protein. p16 also is known to regulate the function of CDK6.

p16^{INK4} belongs to a newly described class of CDK-inhibitory proteins that also includes p16^{B}, p19, p21^{WAF1}, and p27^{KIP1}. The p16^{INK4} gene maps to 9p21, a chromosome region frequently deleted in many tumor types. Homozygous deletions and mutations of the p16^{INK4} gene are frequent in human tumor cell lines. This evidence suggests that the p16^{INK4} gene is a tumor suppressor gene. This interpretation has been challenged, however, by the observation that the frequency of the p16^{INK4} gene alterations is much lower in primary uncultured tumors than in cultured cell lines (Caldas et al., 1994; Cheng et al., 1994; Hussussian et al., 1994; Kamb et al., 1994; Kamb et al., 1994; Mori et al., 1994; Okamoto et al., 1994; Nobori et al., 1995; Orlow et al., 1994; Arap et al., 1995). Restoration of wild-type p16^{INK4} function by transfection with a plasmid expression vector reduced colony formation by some human cancer cell lines (Okamoto, 1994; Arap, 1995).

Other genes that may be employed according to the present invention include Rb, mda-7, APC, DCC, NF-1, NF-2, WT-1, MEN-I, MEN-II, zac1, p73, VHL, MMAC1/PTEN, DBCCR-1, FCC, rsk-3, p27, p27/p16 fusions, p21/p27 fusions, anti-thrombotic genes (e.g., COX-1, TFP1), PGS, Dp, E2F, ras, myc, neu, raf, erb, fms, trk, ret, gsp, hst, abl, E1A, p300, genes involved in angiogenesis (e.g., VEGF, FGF, thrombospondin, BAI-1, GDAIF, or their receptors) and MCC.

Apoptosis, or programmed cell death, is an essential process in cancer therapy (Kerr et al., 1972). The Bc1-2 family of proteins and ICE-like proteases have been demonstrated to be important regulators and effectors of apoptosis in other systems. The Bc1-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bc1-2 protein now is recognized to be a member of a family of related proteins, which can be categorized as death agonists or death antagonists.

Members of the Bc1-2 that function to promote cell death such as, Bax, Bak, Bik, Bim, Bid, Bad and Harakiri, are contemplated for use in combination with syndecan-1 peptides or an analog thereof in treating cancer.

V. Surgery

It is further contemplated that a surgical procedure may be employed in the present invention. Approximately 60% of persons with cancer will undergo surgery of some type, which includes preventative, diagnostic or staging, curative and palliative surgery. Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Syndecan treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

vi. Hormonal Therapy

Hormonal therapy may also be used in conjunction with the syndecan-1 peptides or analog thereof as in the present invention, or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

vii. Other agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adhesion, or agents that increase the sensitivity of the hyperproliferative cells to apoptotic inducers. Immunomodulatory agents include tumor necrosis factor; interferon alpha, beta, and gamma; IL-2 and other cytokines; F42K and other cytokine analogs; or MIP-1, MIP-1β, MCP-1, RANTES, and other chemokines. It is further contemplated that the upregulation of cell surface receptors or their ligands such as Fas/Fas ligand, DR4 or DR5/TRAIL would potentiate the apoptotic inducing abilities of the present invention by establishment of an autocrine or paracrine effect on hyperproliferative cells. Increased intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyperproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

### VIII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### MDA-MB-231 Cell Spreading on VN is Disrupted by Soluble Recombinant S1ED

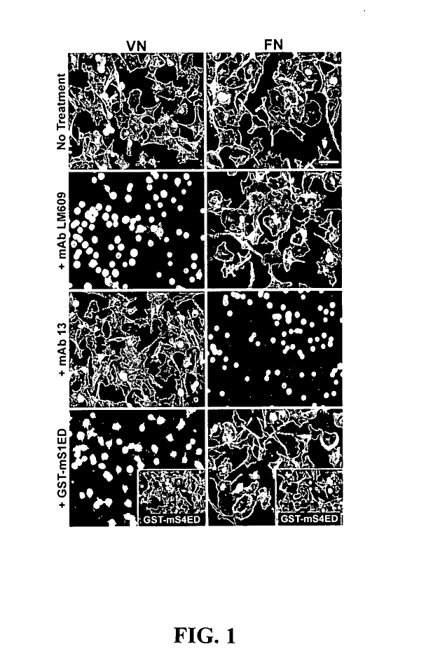

MDA-MB-231 human mammary carcinoma cells, plated in the absence of serum, adhere to and spread (˜15-20 min after plating) on wells coated with either 10 μg/mL VN or FN (FIG. 1). Cells treated with 30 μg/mL LM609 to block αᵥβ₃ integrins fail to spread. LM609 has no effect on spreading in response to FN; instead, the cells rely on αᵥβ₁ integrin to respond to FN as spreading is blocked by either 25 μg/mL mAb 13 (FIG. 1; Mould et al., 1996) or mAb 16 (unpublished data; Akiyama et al., 1989). The Inventors previous studies have shown that αᵥβ₃integrins are essential for signaling when these cells are adherent to Sdc-1-specific antibody, suggesting a collaboration between these two adhesion receptors even in the absence of an integrin ligand; this collaboration between Sdc-1 and αᵥβ₃integrins is disrupted by the addition of recombinant GST-mS1ED (Beauvais and Rapraeger, 2003). To test whether a similar Sdc-1/αᵥβ₃ collaboration is at work when cells are bound to matrix, cells were plated on either VN or FN in the presence of 20 μM recombinant GST-mS1ED. This treatment blocks cell spreading on VN but has no effect on cell spreading on FN (FIG. 1). Treatment with either GST alone (unpublished data) or GST-mS4ED has no effect on spreading on either ligand (FIG. 1, insets).

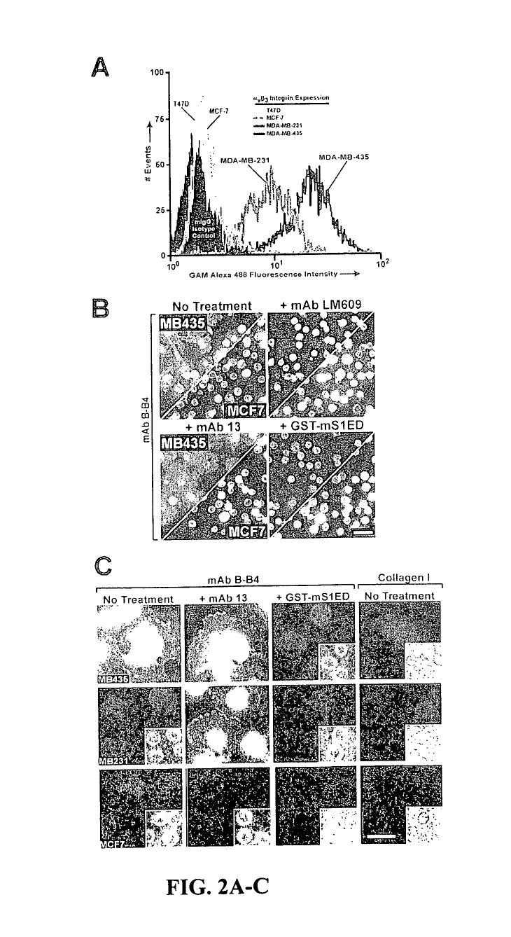

Sdc-1 adhesion-mediated cell spreading correlates with αᵥβ₃ integrin expression and activity. To test whether the αᵥβ₃ integrin's dependence on Sdc-1 extends to other carcinoma cells, the inventors screened a panel of human carcinoma cells with mAb LM609 using FACS analysis. MDA-MB-435 cells exhibit higher αᵥβ₃ expression than MDA-MB-231 cells, while MCF-7 cells are negative for this integrin (FIG. 2A). To test the collaboration between Sdc-1 and αᵥβ₃ integrin when cells are adherent via Sdc-1, MDA-MB-435 and MCF-7 cells were plated on wells coated with hS1-specific mAb B-B4. While αᵥβ₃-positive MDA-MB-435 cells spread on this antibody substratum, their spreading is blocked (similar to results obtained in the MDA-MB-231; Beauvais and Rapraeger, 2003) by treatment with either mAb LM609 (added either before plating or 30 min after plating, when cells have already begun to spread) or GST-mS1ED protein (which is not recognized by mAb B-B4). MCF-7 (FIG. 2B) and T47D (unpublished data) cells, which are αᵥβ₃-negative, bind to mAb B-B4 but fail to spread.

To verify that cell spreading induced upon Sdc-1 ligation correlates with αᵥβ₃activation, the inventors relied upon binding of the ligand-mimetic WOW1 mouse Fab—a probe that detects activated, but unligated αᵥβ₃ integrin. MDA-MB-435 cells adherent to mAb B-B4 display strong binding of WOW1 both centrally and within puncta at the spreading margin of cells (FIG. 2C). In contrast, WOW1 fails to bind to cells treated with 20 μM GST-mS1ED and this correlates with the failure of the cells to spread (phase insets). WOW1 also binds to MDA-MB-231 cells adherent to mAb B-B4 and pre-treated with mAb 13 prior to plating; treatment with mAb 13 relieves a negative β₁-β₃ integrin cross-talk mechanism active in the MDA-MB-231 cells thus allowing for αᵥβ₃ integrin activation (Beauvais and Rapraeger, 2003). Like the MDA-MB-435 cells, WOW1 staining is also lost from the MDA-MB-231 cells when cells are treated with 20 μM GST-mS1ED. As a control, WOW1 fails to bind MDA-MB-435 or MB-231 cells adherent and spread on COLI, a β₁ integrin specific ligand. This agrees with growing evidence that activation of certain β₁ integrins can downregulate αᵥβ₃ integrin activity in a number of cell lines (Beauvais and Rapraeger, 2003; Gonzalez et al., 2002; Kim et al., 2000; Kiosses et al., 2001). The inventors do not see WOW1 binding to the αᵥβ₃-negative MCF-7 cells.

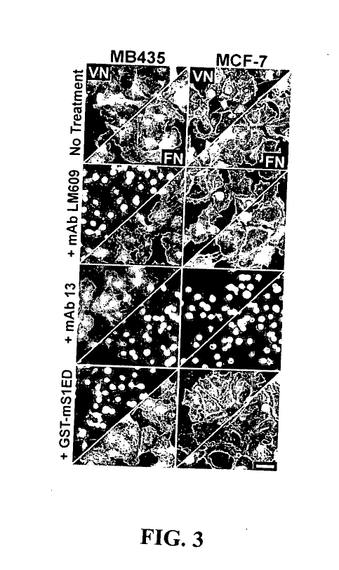

MDA-MB-435, but not MCF-7, human carcinoma cells display functional coupling of Sdc-1 and αᵥβ₃ integrin on VN. To test whether activation of αᵥβ₃ integrin activation on matrix is also functionally coupled to Sdc-1, MDA-MB-435 and MCF-7 cells were plated on either VN or FN (FIG. 3). MDA-MB-435 cells spread on VN and require αᵥβ₃ integrins for this activity as spreading is blocked by mAb LM609. Although the MCF-7 cells spread on VN, this spreading is unaffected by LM609; these cells rely instead on αᵥβ₁ integrins as spreading is blocked by either mAb 13 (FIG. 3) or αᵥ-specific mAb M9 (unpublished data; de Vries et al., 1986). Neither cell type utilizes αᵥβ₃ to respond to FN, rather both utilize αᵥβ1 integrins that are blocked by either mAb 13 (FIG. 3) or mAb 16 (unpublished data).

To test whether GST-mS1ED specifically blocks αᵥβ₃ integrin-dependent spreading or acts as a general inhibitor to all αᵥ integrin heterodimers, MDA-MB-435 and MCF-7 cells were plated on VN in the presence of 20 μM GST-mS1ED. Only the MDA-MB-435 cells fail to spread in response to VN after treatment with GST-mS1ED, although they retain their ability to spread on FN. This effect appears specific for S1ED as treatment with GST alone or GST-mS4ED (unpublished data) has no effect.

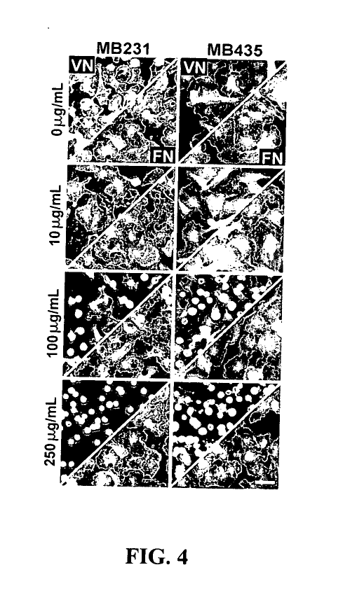

Polyclonal S1ED antibodies disrupt αᵥβ₃ integrin-dependent cell spreading and migration on VN. To target the syndecan directly, MDA-MB-231 and MB-435 (αᵥβ₃-positive) cells were treated with S1ED-specific polyclonal antibodies (pAb) prior to plating. The pAbs recognize Sdc-1 on blots and live cells, but fail to recognize other syndecan family members (unpublished data). The cells display a dose-dependent inhibition in VN-dependent cell spreading over a pAb concentration range of 10-250 μg/mL (FIG. 4A). The number of spread cells on VN was reduced from 92±6% in the absence of pAb to 30±4% at 100 μg/mL pAb with almost complete inhibition (≦7%) for both cell types at 250 μg/mL. Note that the treatment of either cell type with pAb does not alter their spreading in response to FN. The relatively high pAb concentration required to achieve full inhibition of spreading may indicate that the “blocking” antibody is a relatively minor fraction of the pAb mix. Treatment of cells with 250 μg/mL of anti-GST pAb has no effect on cell spreading on either matrix ligand (unpublished data).

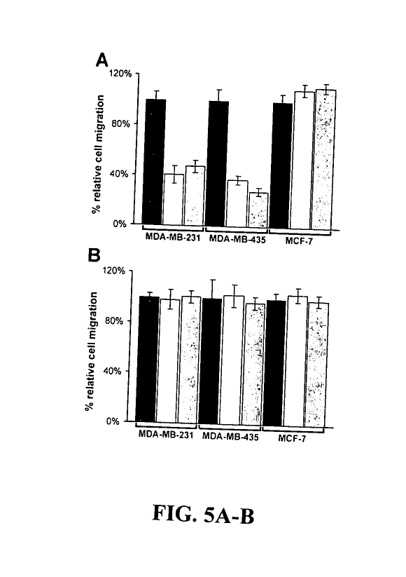

To test the role of the S1ED in αᵥβ₃ integrin dependent signaling in another functional assay, cells were examined for their ability to migrate across VN or FN-coated filters in the presence or absence of S1ED pAb (250 μg/mL) or GST-mS1ED recombinant protein (20 μM). Migration of treated MDA-MB-231 and MB-435 cells across VN is suppressed 2- to 4-fold relative to untreated controls (FIG. 5A). MCF-7 cell migration across VN, which is αᵥβ₁ dependent, is unaffected by either treatment. Further, neither inhibitor has any effect on cell migration across FN in any of the three cell types tested (FIG. 5B).

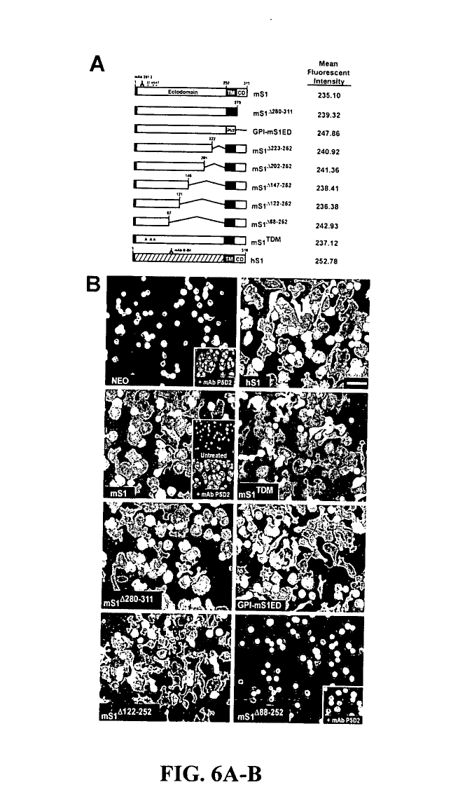

Activity resides within the ectodomain of the Sdc-1 core protein. To confirm that the S1ED is necessary and sufficient for the activation of αᵥβ₃ integrin-dependent cell spreading, MDA-MB-231 cells were transfected with mS1 expression constructs (FIG. 6A). Populations of high-expressing clones were sorted by FACS analysis using mAb 281.2, an antibody that selectively recognizes mS1, to ensure comparable levels of expression. Cells were then plated on Sdc-1 specific antibodies to assess their ability to spread in response to Sdc-1 ligation.

NEO (pcDNA3 empty vector) cells (FIG. 6B) adherent to hS1-specific antibody mAb B-B4 fail to spread unless treated with mAb β₅D2, a β₁ integrin neutralizing antibody (NEO, inset), that relieves an a₂P,-dependent repression of αᵥβ₃ integrins (Beauvais and Rapraeger, 2003). To test the activity of the mS1 constructs, cells were plated on mAb 281.2. Interestingly, cells expressing full length mS1 spread (FIG. 6B, mS1) without prior inhibition of β₁. This response is not unique to mS1 as hS1-overexpressors (FIG. 6B, hS1), when plated on mAb B-B4, also spread in the absence of β₁ integrin blockade suggesting that overexpression of Sdc-1 overcomes the negative β₁-β₃ integrin cross-talk mechanism in these cells. Intriguingly, mS1-overexpressing cells fail to spread on mAb B-B4 unless cells are treated with the β₁ blocker (insets, mS1) mimicking the response of NEO cells plated on a similar substratum (FIG. 6B, NEO). Thus, endogenous hS1 and ectopic mS1 appear to act independently of each other and the cells respond only to the ligated Sdc-1.

To identify the properties of Sdc-1 required to regulate αᵥβ₃ integrin activity, cells expressing mS1 mutants were plated on a substratum of mAb 281.2. A mutant that lacks its HS chains (mS1^{TDM}) retains its ability to spread. Cells expressing a Sdc-1 construct that lacks either its cytoplasmic domain (mS₁^{Δ280-311}) or both its TM and cytoplasmic domains (GPI-mS1ED) retain their ability to spread—confirming that activity resides in the S1ED. Mutants with progressively larger ED deletions (mS1²²³⁻²⁵², mS1^{Δ202-252} and mS1^{Δ147-252}) all retain activity (unpublished data) as does a Sdc-1 construct (mS1¹²²⁻²⁵²) that lacks 131 amino acids located between the HS attachment sites and the TM domain. However, cells expressing a mutant (mS1^{Δ88-252}) that lacks 34 additional amino acids, fail to spread and spreading cannot be rescued by treatment with mAb P5D2 (inset, mS1^{Δ88-252})—a treatment that would have otherwise enhanced αᵥβ₃integrin activation (inset, NEO).

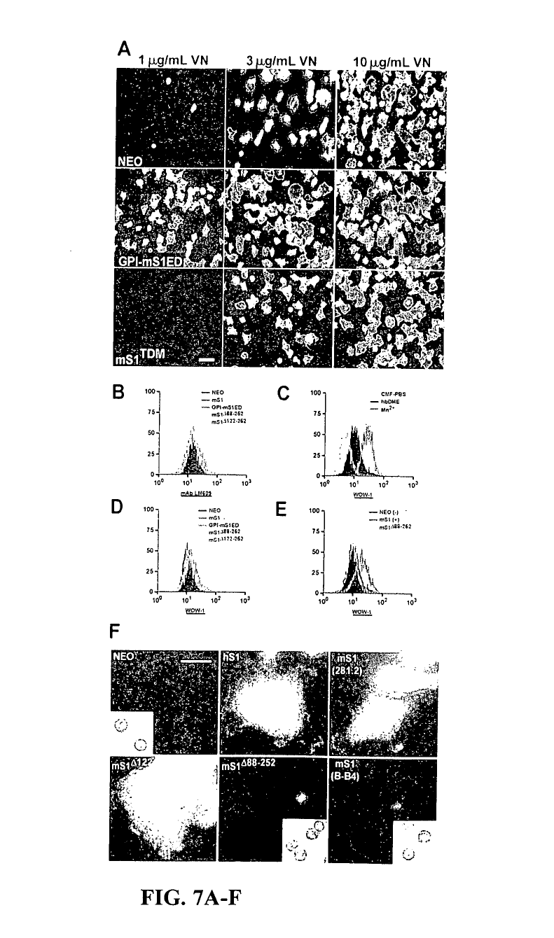

Overexpression and ligation of Sdc-1 “primes” cells to spread in response to VN. To test whether overexpression of Sdc-1 enhances VN recognition via the αᵥβ₃integrin, cell attachment and spreading were assessed on wells containing increasing concentrations of VN (1, 3 and 10 μg/mL) (FIG. 7A). NEO control cells largely fail to bind to wells coated with 1 μg/mL VN and display only modest spreading in response to 3 μg/mL. Full adhesion and spreading is not achieved until cells encounter high concentrations of VN (10 μg/mL). In contrast, cells overexpressing the S1ED (GPI-mS1ED) attach and spread on low VN concentrations and this response increases with higher concentrations of VN. However, this response is dependent on Sdc-1's engagement of the matrix as cells expressing mS1^{TDM} (which lacks its HS chains) mimic the response of NEO control cells.

These results correlate with the induction in αᵥβ₃activation observed when cells are adherent to Sdc-1 antibody (FIG. 2C) and suggest that αᵥβ₃activation depends on Sdc-1 being engaged by ligand. To test this, integrin activation and expression was examined in Sdc-1 overexpressing cells in suspension (±mS1 clustering) and when adherent to Sdc-1 antibody. On suspended cells when mS1 is not clustered, the inventors detect no increase in αᵥβ₃ activity (WOW1, FIG. 7D). However, when mS1 is clustered using mAb 281.2, although the inventors detect no change in αᵥβ₃expression (mAb LM609, FIG. 7B), the inventors do detect a demonstrable increase in αᵥβ₃ activity with either mS1 or GPI-mS1ED (unpublished data), but not with mS1^{Δ88-252} (WOW1, FIG. 7E). Activation of the integrin with 1.0 mM Mn²⁺ (Lin et al., 1997; Pampori et al., 1999; Smith et al., 1994) is shown for comparison (FIG. 7C); this contrasts with the diminished WOW1 binding observed following inactivation of the integrin by the removal of divalent cations (CMF-PBS, FIG. 7C).

Although enhanced Sdc-1 expression has no effect on αᵥβ₃ activation levels on suspended cells (FIG. 7D), a different result is obtained when the cells are assessed in antibody-based adhesion assays (FIG. 7F). Cells overexpressing hS1 or mS1 stain positively with WOW1 when adherent to their respective Sdc-1-specific antibodies, while NEO cells bound to B-B4 display no significant WOW1 binding. However, cells overexpressing mS1 fail to bind WOW1 when adherent via their endogenous hS1 (B-B4) indicating again that the syndecan must be ligated to efficiently activate the αᵥβ₃ integrin. Cells overexpressing mS1^{Δ122-252}, a S1ED mutant that retains its ability to spread in response to Sdc-1 (FIG. 6B), also display positive staining for WOW1, but cells overexpressing mS1^{Δ88-252}, a mutant which fails to signal spreading, do not.

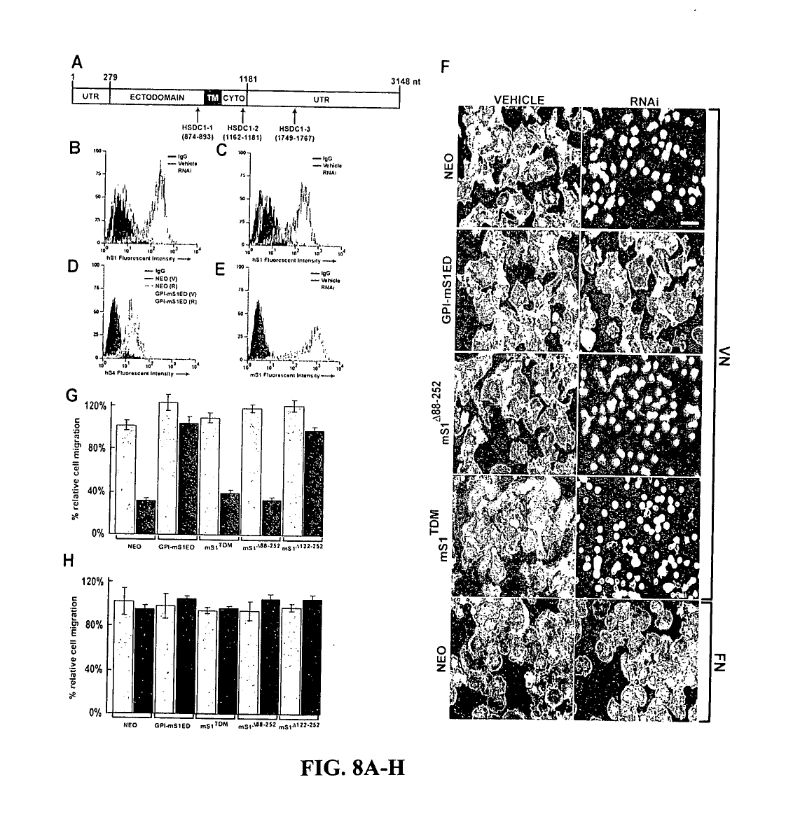

Downregulation of Sdc-1 expression by siRNA disrupts cell spreading and migration in response to VN. To test the activity of the mS1 mutants on matrix ligands, the expression of the endogenous hS1 needs to be blocked. Thus, cells expressing mS1 constructs were transfected with siRNA designed to specifically target hS1 (FIG. 8A). Transfection with siRNA efficiently silences hS1 (greater than 90% reduction) in both NEO vector-control cells (FIG. 8B) and in cells expressing mS1 constructs (GPI-mS1ED provided as a representative result; FIG. 8C). Importantly, hS1 siRNA affects neither mS1 expression (FIG. 8E) nor the expression of hS4 in either NEO vector control or mS1-expressing cells (FIG. 8D). In addition, hS1 siRNA has no effect on the expression levels of either αᵥβ₃ or β₁ integrins as determined by FACS (unpublished data).

NEO vector control cells lacking hS1 fail to spread in response to VN, but are able to spread on FN. Cell spreading on VN is recovered by expression of GPI-mS1ED (FIG. 8F) and mS^{Δ122-212} (unpublished data). It is unlikely that the siRNA treatment has any non-specific cellular effects since spreading is specifically rescued by expression of GPI-mS1ED. However, spreading is not recovered in cells expressing mS1^{Δ88-252}, the S1ED mutant that fails to signal spreading in Sdc-1 antibody-based adhesion assays. Cells expressing mS1^{TDM}, a mutant unable to engage the matrix, also fail to spread on VN confirming that Sdc-1 mediated adhesion is required for full αᵥβ₃ integrin activity.

To test the effects of hS1 silencing on cell migration, the migration of hS1 siRNA transfected cells was examined on VN or FN-coated filters. Migration of the NEO cells across VN is reduced approximately 3-fold by siRNA relative to untreated controls (FIG. 8G). In addition, cell migration in response to VN is rescued in the siRNA-treated cells by expression of GPI-mS1ED or mS1^{Δ122-252}, but not by mS1^{TDM} or mS1^{Δ88-252}. None of the cells display any defects in their ability to migrate in response to FN (FIG. 8H).

### Example 2

### Syndecan-1 Regulates Activity of the αᵥβ₅ Integrin on B82L Fibroblasts

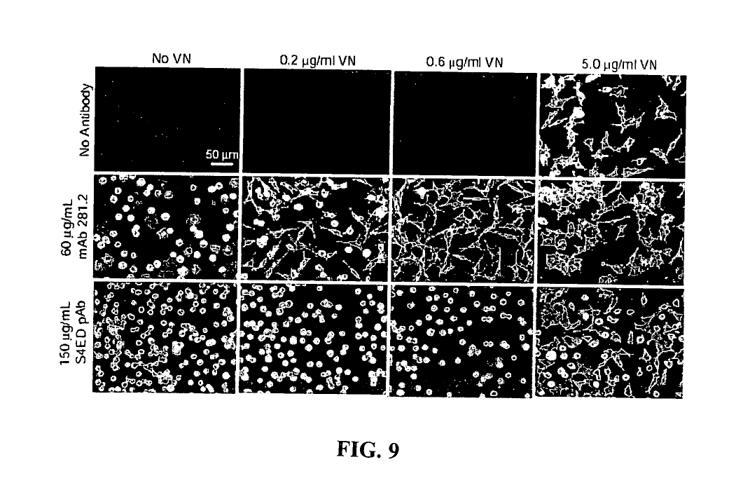

Syndecan-1 Mediates Spreading in B82L Fibroblasts on VN and FN. B82L fibroblasts require relatively high concentrations of VN or FN in order to attach to substrata coated with these matrix ligands. This suggests that the integrin necessary for their recognition is not in an activated state. To test whether ligation of syndecan can enhance this activation, as it does with the αᵥβ₃ integrin, B82L fibroblasts were added to wells on which mn=Ab 281.2, which is specific for mouse Sdc-1, was co-coated with limiting dilutions of VN (FIG. 9) and FN (not shown). In the absence of the syndecan-specific antibody, B82L cell binding and spreading requires relatively high plating concentrations of VN (5 μg/mL) or FN (60 μg/mL). Even a two-.fold reduction in this amount completely abolishes binding (data not shown); thus, no binding is observed when only 0.2 or 0.6 μg/mL VN (FIG. 9) or 1 or 3 μg/mL FN (not shown) is provided. However, cells in which Sdc-1 is engaged by plating on mAb 281.2 will spread with a fusiform morphology when as little as 0.2 μg/mL VN or 1 μg/mL FN, a level that by itself is insufficient to sustain adhesion, is co-plated with the antibody.

One possible explanation for increased sensitivity to these matrix ligands is that the Sdc-1 bound to antibody simply tethers cells to the substratum and in this manner alone facilitates their interaction with low levels of matrix ligands to which cells would normally not adhere. To test this possibility, cells were plated on substrata comprised of increasing amounts of either VN or FN co-coated with an antibody directed against the ectodomain of Sdc-4, a syndecan that is expressed at equal levels to Sdc-1 (Ott and Rapraeger, 1998). Because of their anchorage to the antibody, cell binding is seen either with antibody alone, or with the mixed antibody and matrix ligand substrata (FIG. 9). However, the cells fail to spread, either on Sdc-4 antibody alone, or when the antibody is supplemented with low concentrations of either VN or FN. The cells spread only when VN or FN reaches a concentration that promotes adhesion and spreading on its own, e.g., 5 μg/mL VN and 60 μg/mL FN (FIG. 9). Thus, the Sdc-1 must be fulfilling more than just a simple tethering role.

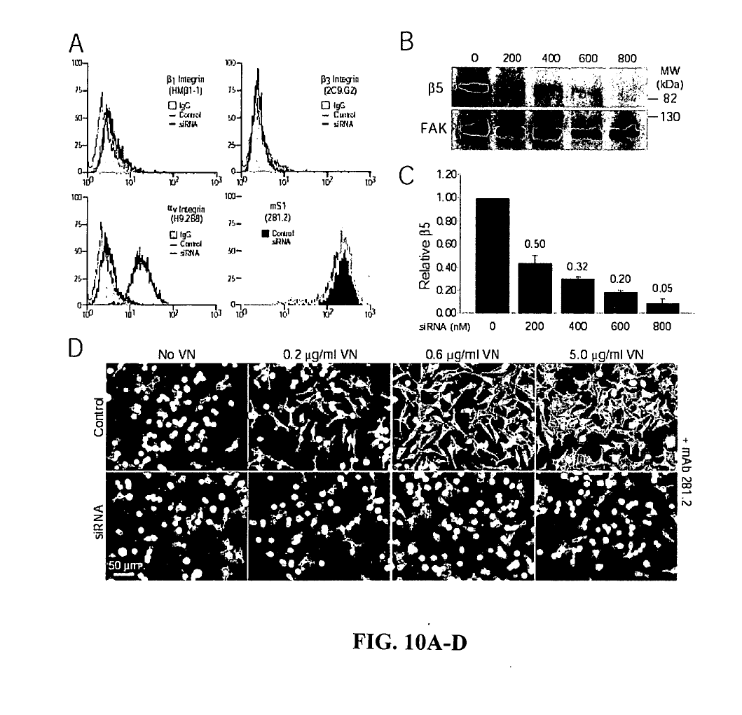

Response to VN and FN requires the αᵥβ₅ Integrin. The αᵥβ₁ and αᵥβ₃integrins are expressed on fibroblasts and are well known to act as VN or FN receptors (Sanders et al., 1998). The αᵥβ₅ integrin has also been shown to be involved in fibroblast cell spreading on VN and FN (Pasqualini et al., 1993; van Leeuwen et al., 1996). Analysis of integrin expression of the B82L cells by flow cytometry shows that they express the a, integrin subunit, but relatively low amounts of the β₁ integrin subunit and little or no β₃ (FIG. 10A). Furthermore, B82L fibroblasts treated with β₁ or β₃ integrin inhibitory antibodies, mAb HMβ1-1 (Noto et al., 1995) and mAb 2C9.G2 (Yasuda et al., 1995) respectively, or both antibodies together, show no effect on adhesion or spreading on Sdc-1 antibody plus VN or FN or on high concentrations of matrix ligand alone (data not shown).

These data suggest the αᵥβ₅ integrin as a candidate for Sdc-1 regulation. Unfortunately, neither inhibitory antibodies to the mouse β₅ subunit nor antibodies amenable for use in flow cytometry are currently available. However, Abl926 binds the cytoplasmic domain of the β₅ subunit and western blot analysis of B82L cell lysates shows expression of this integrin subunit (FIG. 10B). To test the role of the αᵥβ₅ integrin in Sdc-1-regulated cell spreading, an siRNA oligonucleotide specific for the mouse β₅ subunit was used to block αᵥβ₅ integrin expression. Transfection of cells with this siRNA reduces αᵥ-containing integrin expression by approximately 90%, as shown by monitoring cc integrin expression by flow cytometry (FIG. 10A). This correlates with a similar reduction in β₅ subunit expression observed on western blots upon treatment with a range of siRNA concentrations (FIG. 10B-C). The siRNA has no effect on mouse Sdc-1 expression or the expression of other integrin β-subunits (FIG. 10A). Finally, it is observed that cells treated with 800 mM siRNA to block β₅ integrin expression fail to respond to either VN or FN when plated on these ligands together with Sdc-1 antibody, even at high concentrations of VN or FN that are sufficient to promote cell spreading without Sdc-1 antibody (FIG. 10D).

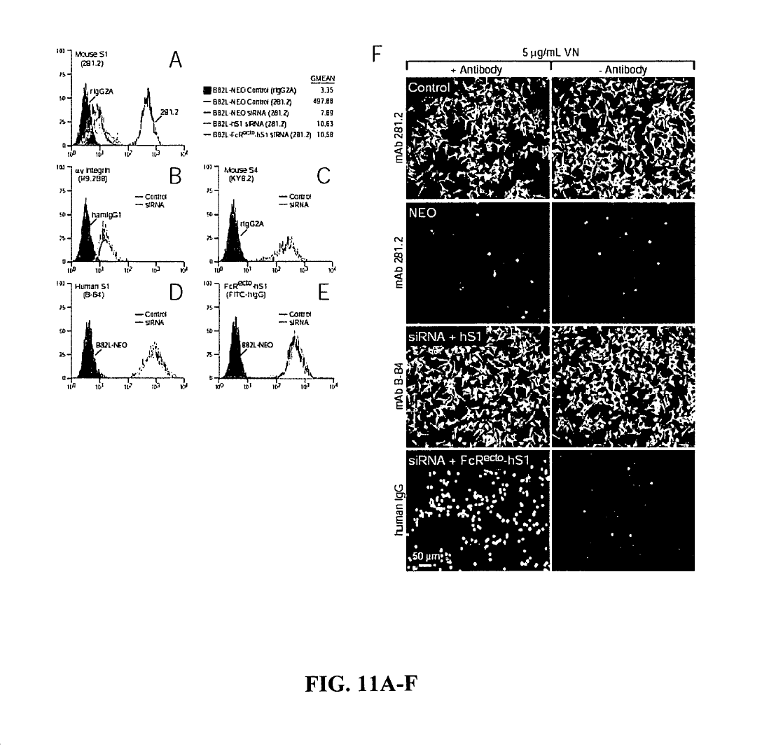

αᵥβ₅-dependent cell attachment and spreading requires the Sdc-1 ectodomain. The loss of Sdc-1-regulated cell spreading in cells transfected with β₅ siRNA indicates that the αᵥβ₅ integrin is the αᵥ-bearing integrin targeted by the syndecan. To test what domain(s) of the syndecan is required for this activity, endogenous mouse Sdc-1 expression was silenced with mouse-specific siRNA and replaced by expression of human Sdc-1 constructs (FIG. 11A). Transfection with siRNA efficiently silences mouse Sdc-1 by ˜98% as indicated by FACS analysis. Importantly, the mouse Sdc-1-siRNA does not affect the expression of αᵥβ₅, as indicated by western blotting (data not shown) and FACS analysis of the αᵥ integrin (FIG. 11B), nor does it affect the expression of mouse Sdc-4 (FIG. 11C) or the human Sdc-1 constructs (FIG. 11D,E).

In contrast to parental cells, B82L vector-control cells (NEO) transfected with mouse Sdc-1-siRNA fail to attach and spread to wells coated with either 5 μg/mL of VN alone (FIG. 11F, right column) or a mixed substratum of 5 μg/mL of VN plus 60 μg/mL of mAb 281.2 (FIG. 11F, left column). The failure of NEO cells to engage the mixed substratum is indicative of the efficient blockade of mouse Sdc-1 expression by the siRNA. It is unlikely that the mouse Sdc-1-siRNA treatment has any non-specific cellular effects since spreading on VN is specifically rescued by the expression of full-length human Sdc-1 (hS1, FIG. 11F). Spreading of the B82L-hS1 cells is indistinguishable from parental cells. However, spreading on VN is not recovered in cells expressing the FcR^{ecto}-hS1 chimera—a construct in which the syndecan's ectodomain is replaced by the ectodomain of the human Fcγ receptor Ia (FcR^{ecto}-hS1)—regardless of whether the cells are plated on VN alone, or VN supplemented with 60 μg/mL of hIgG to engage the FcR^{ecto}-hS1 construct. These results suggest that αᵥβ₅ integrin activity is dependent on Sdc-1 expression and that the syndecan's ectodomain regulates such activity.

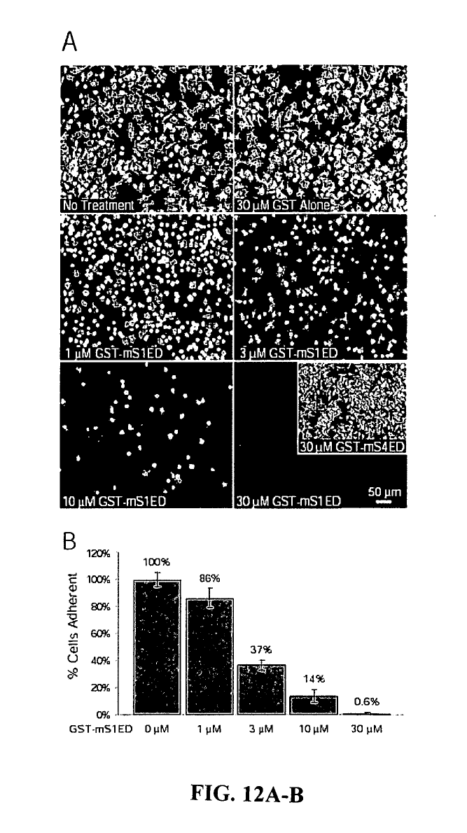

Recombinant Sdc-1 ectodomain competitively blocks αᵥβ₅ integin activity. To test whether soluble Sdc-1 ectodomain will compete with the syndecan activation of the αᵥβ₅ integrin, B82L cells were plated on 5 μg/mL VN in the presence of a recombinant GST fusion protein containing either the ectodomain of mouse Sdc-1 (GST-mS1ED) or human Sdc-1 (GST-hS1ED; data not shown). Because the fusion protein is derived from bacteria, it does not contain attached GAG chains. GST-mS1ED and hS1ED display similar activity; competition occurs at the lowest concentration tested (1 μM) with increasing blockade of cell attachment and spreading over a concentration range of 1-30 μM of S1ED (FIG. 12A, B). Similar results are obtained with GST-mS1ED and B82L-hS1 cells attached to a mixed substratum of mAb B-B4 and 1 μg/mL VN (data not shown). It should be noted that mS1ED is not recognized by the human specific mAb B-B4 and thus does not compete for human Sdc-1 engagement of the antibody substratum. Competition with 30 μM GST alone is without effect in all cases, as is competition with identical concentrations of recombinant GST-mS4ED indicating that competition for syndecan-regulated αᵥβ₅ activity is S1ED-specific.

### Example 3

### Syndecan-1 Regulates the αᵥβ3 and αᵥβ5 Integrins on Endothelial Cells

Numerous studies have shown that endothelial cells rely on the activity of the αᵥβ3 and αᵥβ5 integrins, especially during proliferation and survival during growth factor-induced angiogenesis. Human aortic endothelial cells (HAEC) express abundant amounts of the αᵥβ3 integrin and somewhat less of the αᵥβ5. The cells also express a high level of the β1 integrin subunit, which is capable of assembling with the αᵥ subunit to form the αᵥβ1 integrin. All three of these integrins are potential vitronectin receptors and it is thus not surprising that these endothelial cells bind and spread on vitronectin.

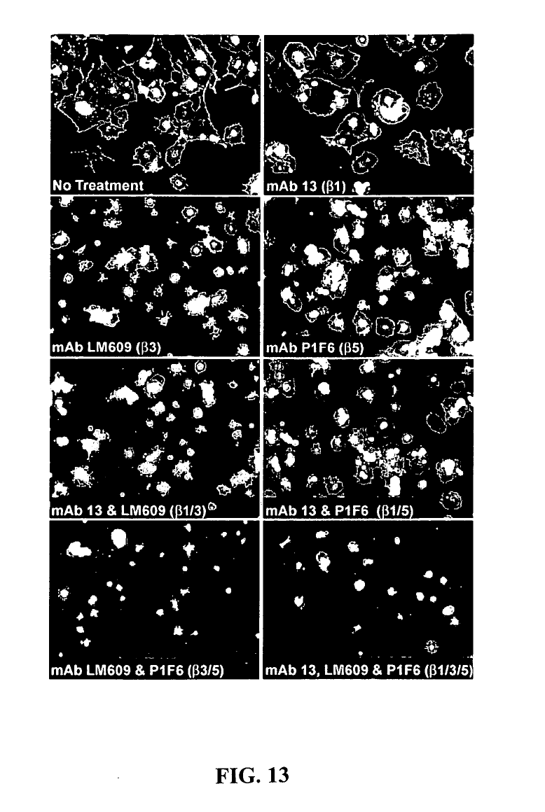

To assess the contribution of each integrin, the inventors plated HAEC cells on vitronectin in the presence of inhibitory antibodies to the β1 integrins (mAb13), the αᵥβ3 (LM609) and the αᵥβ5 (P1F6). Although inhibiting the αᵥβ1 integrin had little or no effect, the HAECs continued to attach and spread on VN unless both αᵥβ3 and αᵥβ5 were inhibited (FIG. 13).

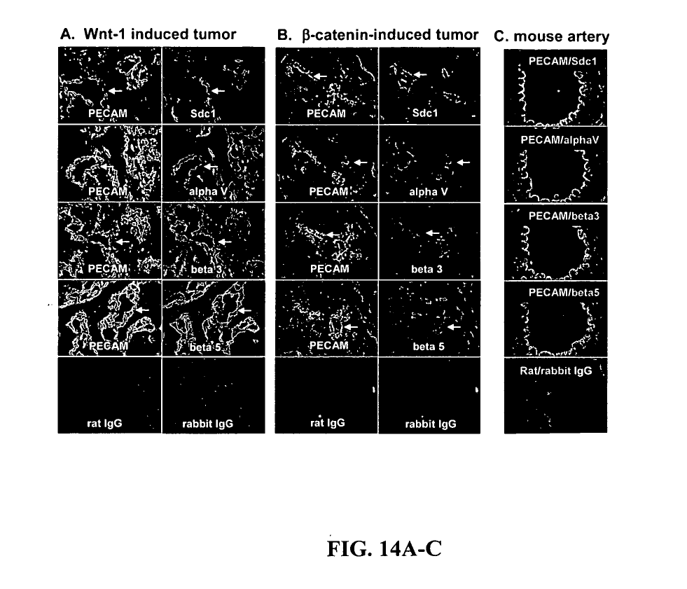

Next, the inventors examined the expression of Sdc-1. Flow cytometry of cells using the human specific Sdc-1 antibody B-B4 showed that the HAECs have a significant cell surface population of Sdc-1. They also examined the expression of Sdc-1 during tumor-induced angiogenesis, using mammary carcinomas arising in mice co-expressing either the wnt1 oncogene, or the β-catenin oncogene. Staining for Sdc-1 showed that is was highly expressed in the angiogenic tumor vasculature of both tumors, and was co-expressed at this site with the αᵥβ3 and αᵥβ5 integrins (FIGS. 14A-C).

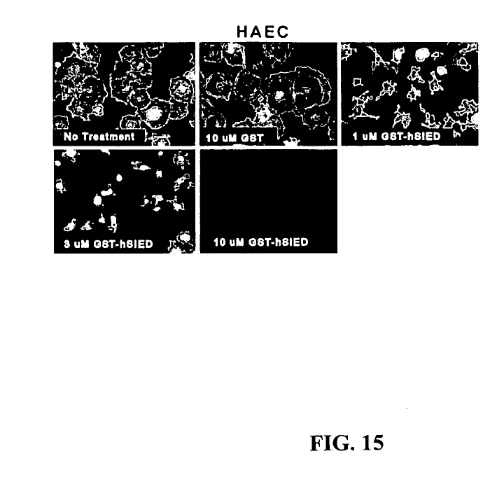

The next test was whether agents that target Sdc-1, especially competition with recombinant Sdc-1 ectodomain, would block the activity of both or either integrin. Indeed, the HAEC cell spreading on VN was blocked by the soluble S1ED, at a concentration similar to that of the αᵥβ3 in mammary carcinoma and that of the αᵥβ5 integrin in B82L fibroblasts (FIG. 15). Since the inventors' work with inhibitory integrin antibodies demonstrated that both integrins must be blocked on these cells in order to block cell spreading, they thus conclude that the recombinant protein must disrupt signaling by both of these integrins.

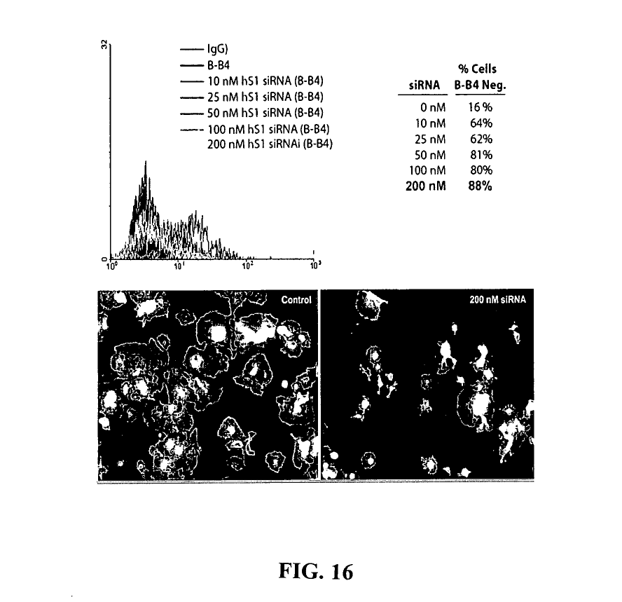

The inventors also performed an analysis using mouse aortic endothelial cells, which express Sdc-1, and found that they are exquisitely sensitive to competition by the Sdc-1 ectodomain, with spreading on VN blocked using 1 μM competitor. Secondly, they targeted Sdc-1 expression cells using siRNA. Preliminary experiments demonstrated that at 100 mM siRNA, which silences Sdc-1 expression by 80%, the spreading of the HAECs on VN is also disrupted (FIG. 16).

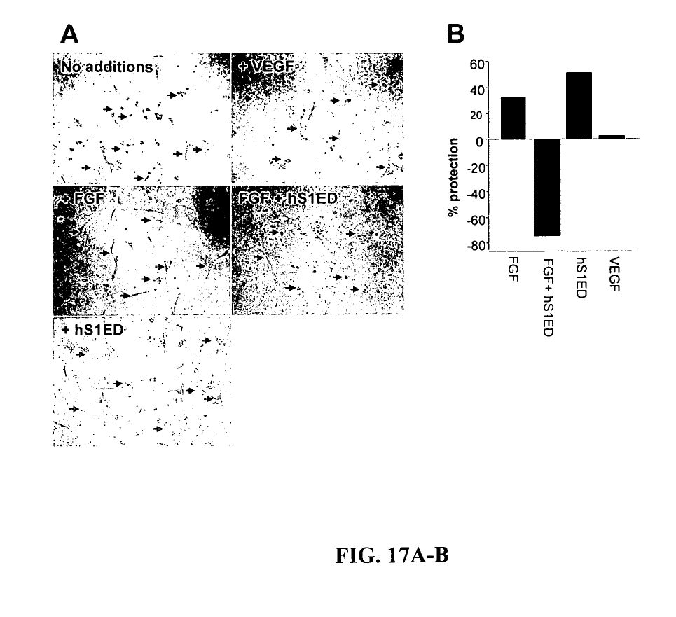

Finally, the inventors performed used human microvascular endothelial cells (HMECs), which also express Sdc-1, as well as the αᵥβ3 and αᵥβ5 integrins, to test the theory that endothelial cells undergoing angiogenesis in response to fibroblast growth factor, a common angiogenic agent, will enter apoptosis and die unless the FGF signal is accompanied by a coordinate signal from the αᵥβ3 integrin. The inventors hypothesis was that recombinant ectodomain of Sdc-1 would block the signal from the αᵥβ3 integrin and induce apoptosis in the presence of FGF. HMEC cells grown in low serum conditions spontaneously enter apoptosis. Cells under this condition were treated with 100 ng/ml FGF and showed a reduction in cell death, as anticipated (FIGS. 17A-B). However, cells treated with FGF in combination with 30 μM recombinant Sdc-1 ectodomain did not experience the protective effect of FGF. In fact, treatment with FGF together with the soluble ectodomain protein induced apoptosis beyond that of the low serum condition alone (FIGS. 17A-B).

Thus, Sdc-1 on endothelial cells appears to be a critical regulator of the αᵥβ₃and αᵥβ₅ integrins and this activity, and thus the activity of the integrins themselves is blocked by soluble S1ED. This mimics the inhibition observed and published with the human mammary carcinoma cells. Furthermore, is suggests the the soluble S1ED will not only prevent the activity of these two integrins in cell migration and proliferation, but will also promote cell death of endothelial cells undergoing angiogenesis. Thus, the protein is a promising inhibitor of angiogenesis caused by tumors and other human diseases.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,196,265
U.S. Pat. No. 4,554,101
U.S. Pat. No. 4,683,202
U.S. Pat. No. 5,302,523
U.S. Pat. No. 5,322,783
U.S. Pat. No. 5,384,253
U.S. Pat. No. 5,399,363
U.S. Pat. No. 5,440,013
U.S. Pat. No. 5,446,128
U.S. Pat. No. 5,464,765
U.S. Pat. No. 5,466,468
U.S. Pat. No. 5,475,085
U.S. Pat. No. 5,538,877
U.S. Pat. No. 5,538,880
U.S. Pat. No. 5,543,158
U.S. Pat. No. 5,550,318
U.S. Pat. No. 5,563,055
U.S. Pat. No. 5,580,859
U.S. Pat. No. 5,589,466
U.S. Pat. No. 5,610,042
U.S. Pat. No. 5,618,914
U.S. Pat. No. 5,641,515
U.S. Pat. No. 5,656,610
U.S. Pat. No. 5,670,155
U.S. Pat. No. 5,672,681
U.S. Pat. No. 5,674,976
U.S. Pat. No. 5,702,932
U.S. Pat. No. 5,710,245
U.S. Pat. No. 5,736,524
U.S. Pat. No. 5,780,448
U.S. Pat. No. 5,789,215
U.S. Pat. No. 5,840,833
U.S. Pat. No. 5,846,225
U.S. Pat. No. 5,846,233
U.S. Pat. No. 5,859,184
U.S. Pat. No. 5,928,906
U.S. Pat. No. 5,929,237
U.S. Pat. No. 5,945,100
U.S. Pat. No. 5,981,274
U.S. Pat. No. 5,994,624
Adams et al., J. Cell Biol., 152:1169-1182, 2001.
Akiyama et al., J. Cell Biol., 109:863-875, 1989.
Albert et al., Nat. Cell Biol., 2:899-905, 2000.
Alexander et al., Nat. Genet., 25:329-332, 2000.
Anttonen et al., Br. J. Cancer, 79:558-564, 1999.
Arap et al., Cancer Res., 55(6):1351-1354, 1995.
Ausubel et al., In: Current Protocols in Molecular Biology, John, Wiley & Sons, Inc, New York, 1994.
Baciu and Goetinck, Mol. Biol. Cell, 6:1503-1513, 1995.
Baichwal and Sugden, In: Gene Transfer, Kucherlapati (Ed.), New York, Plenum Press, 117-148, 1986.
Bakhshi et al., Cell, 41(3):899-906, 1985.
Barany and Merrifield, In: The Peptides, Gross and Meienhofer (Eds.), Academic Press, New York, pp. 1-284 1979.
Barbareschi et al., Cancer, 98:474-483, 2003.
Bayer-Garner et al., J. Cutan. Pathol., 28:135-139, 2001.
Beauvais and Rapraeger, Exp. Cell Res., 286:219-232, 2003.
Beauvais and Rapraeger, Reprod. Biol. Endocrinol., 2:3, 2004.
Beglova et al., Nat. Struct. Biol., 9:282-287, 2002.
Bernfield et al., Annu. Rev. Biochem., 68:729-777, 1999.
Blystone et al., J. Biol. Chem., 271:31458-31462, 1996.
Boettiger et al., J. Biol. Chem., 276:31684-31690, 2001.
Borges et al., J. Biol. Chem., 275:39867-39873, 2000.
Boudreau et al., J. Cell Biol., 139(1):257-264, 1997.
Brooks et al., Cell, 79:1157-1164, 1994.
Brooks et al., J. Clin. Invest., 99:1390-1398, 1997.
Brooks et al., Science, 264:569-571, 1994.
Burbach et al., Exp. Cell Res., 300(1):234-247, 2004.
Burbach et al., Matrix Biol., 22:163-177, 2003.
Caldas et al., Nat. Genet., 8(1):27-32, 1994.
Calderwood et al., J. Biol. Chem., 274:28071-28074, 1999.
Calderwood et al., J. Biol. Chem., 277:21749-21758, 2002.
Capaldi et al., Biochem. Biophys. Res. Comm., 74(2):425-433, 1977.
Carey et al., Exp. Cell Res., 214:12-21, 1994.
Carey et al., J. Cell Biol., 124:161-170, 1994.
Carman and Springer, Curr. Opin. Cell Biol., 15:547-556, 2003.
Charo et al., J. Cell Biol., 111:2795-2800, 1990.
Chen and Okayama, Mol. Cell Biol., 7(8):2745-2752, 1987.
Cheng et al., Cancer Res., 54(21):5547-5551, 1994.
Cleary and Sklar, Proc. Natl. Acad. Sci. USA, (21):7439-7443, 1985.
Cleary et al., J. Exp. Med., 164(1):315-320, 1986.
Colorado et al., Cancer Res., 60(9):2520-2526, 2000.
Conejo et al., Int. J. Cancer, 88:12-20, 2000.
Couchman et al., Int. Rev. Cytol., 207:113-150, 2001.
Coupar et al., Gene, 68:1-10, 1988.
Crescimanno et al., J. Pathol., 189:600-608, 1999.
de Vries et al., Int. J. Cancer, 38:465-473, 1986.
De, et al., J. Biol. Chem., 278:39044-39050, 2003.
Degryse et al., Oncogene, 20:2032-2043, 2001.
Du et al., J. Biol. Chem., 270:26146-26151, 1995.
Elenius et al., J. Cell Biol., 114(3): 585-595, 1991.
Eliceiri and Cheresh, J. Clin. Invest., 103:1227-1230, 1999.
Eliceiri et al., J. Cell Biol., 140:1255-1263, 1998.
Eliceiri, Circ. Res., 89:1104-1110, 2001.
Fechheimer et al., Proc. Natl. Acad. Sci. USA, 84:8463-8467, 1987.
Felding-Habermann and Cheresh, Curr. Opin. Cell. Biol., 5:864-868, 1993.
Felding-Habermann et al., Proc. Natl. Acad. Sci. USA, 98:1853-1858, 2001.
Finnemann, Embo. J., 22:4143-4154, 2003a.
Finnemann, Adv. Exp. Med. Biol., 533:337-342, 2003b.
Fraley et al., Proc. Natl. Acad. Sci. USA, 76:3348-3352, 1979.
Friedlander et al., Science, 270:1500-1502, 1995.
Friedmann, Science, 244:1275-1281, 1989.
Fujimoto et al., J. Biol. Chem., 278:26655-26665, 2003.
Fujiya et al., Jpn. J. Cancer Res., 92:1074-1081,2001.
Gallo et al., J. Invest. Dermatol., 107(5):676-683, 1996.
Gao et al., J. Cell Biol., 135:533-544, 1996.
Giancotti and Ruoslahti, Science, 285:1028-1032, 1999.
Gonzalez et al., Proc. Natl. Acad. Sci. USA, 99:16075-16080, 2002.
Gopal, Mol. Cell Biol., 5:1188-1190, 1985.
Gotte et al., Invest. Ophthal. Visual Sci., 43(4):1135-1141, 2002.
Graham and Van Der Eb, Virology, 52:456-467, 1973.
Granes et al., Exp. Cell Res., 248:439-456, 1999.
Hall et al., Exp. Eye Res., 77:281-286, 2003.
Hansen et al., J. Cell Biol., 126:811-819, 1994.
Harland and Weintraub, J. Cell Biol., 101:1094-1099, 1985.
Harlow and Lane, In: Antibodies, A Laboratory manual, Cold Spring Harbor Laboratory, 1988.
Hermonat and Muzycska, Proc. Natl. Acad. Sci. USA, 81:6466-6470, 1984.
Hirabayashi et al., Tumour Biol., 19:454-463, 1998.
Hollstein et al., Science, 253(5015):49-53, 1991.
Hood et al., J. Cell Biol., 162:933-943, 2003.
Horwich et al., Virol., 64:642-650, 1990.
Hughes et al., J. Biol. Chem., 271:6571-6574, 1996.
Hussussian et al., Nat. Genet., 8(1):15-21, 1994.
Iba et al., J. Cell Biol., 149:1143-1156, 2000.
Ilic et al., J. Cell Biol., 143:547-560, 1998.
Inki and Jalkanen, Ann. Med., 28:63-67, 1996.
Inki et al., Br. J. Cancer, 70:319-323, 1994.
Inki et al., J. Pathol., 172:349-355, 1994.
Izzard et al., Exp. Cell Res., 165:320-336, 1986.
Jenkins et al., J. Biol. Chem., 273:13878-13885, 1998.
Johannesson et al., J. Med. Chem., 42(22):4524-4537, 1999.
Johnson et al., In: Biotech. Pharm., Pezzuto et al. (Eds.), Chapman and Hall, NY, 1993.
Jones et al., J. Oral. Pathol. Med., 26:63-68, 1997.
Kaeppler et al., Plant Cell Reports 9: 415-418, 1990.
Kamb et al., Nat. Genet., 8(1):23-26, 1994.
Karnb et al., Science, 2674:436-440, 1994.
Kamphaus et al., J. Biol. Chem., 275(2):1209-1215, 2000.
Kaneda et al., Science, 243:375-378, 1989.
Kashiwagi et al., J. Cell Biol., 137:1433-1443, 1997.
Kato et al, J. Biol. Chem., 266:3361-3364, 1991.
Kato et al., Mol. Biol. Cell, 6:559-576, 1995.
Kerr et al., Br. J. Cancer, 26(4):239-257, 1972.
Khan et al., J. Biol. Chem., 263:11314-113148, 1988.
Kim et al., J. Biol. Chem., 275:33920-33928, 2000.
Kim et al., Mol. Biol. Cell, 5:797-805, 1994.

Kim et al., Oncogene, 22:826-830, 2003.
Kiosses et al., Nat. Cell Biol., 3:316-320, 2001.
Kirsch et al., J. Biol. Chem., 277:42171-42177, 2002.
Klass et al., J. Cell Sci., 113:493-506, 2000.
Klatka, Eur. Arch. Otorhinolaryngol., 259:115-118, 2002.
Kumar-Singh et al., J. Pathol., 186:300-305, 1998.
Kyte and Doolittle, J Mol Biol, 157(1):105-32, 1982.
Law et al., Nature, 401:808-811, 1999.
Lebakken, and Rapraeger, J. Cell Biol., 132:1209-1221, 1996.
Leppa et al. Cell Regul., 2:1-11, 1991.
Leppa et al., J. Cell Sci., 109:1393-1403, 1996.
Leppa et al., Proc. Natl. Acad. Sci. USA, 89:932-936, 1992.
Levy et al., Br. J. Cancer, 74:423-431, 1996.
Levy et al., Bull. Cancer, 84:235-237, 1997.
Liapis et al., Diagn. Mol. Pathol., 5:127-135, 1996.
Lin et al., J. Biol. Chem., 272:14236-14243, 1997.
Lindberg et al., J. Cell Biol., 134:1313-1322, 1996.
Liu et al., J. Biol. Chem., 273:22825-22832, 1998.
Maeshima et al., J. Biol. Chem., 275(28):21340-21348, 2000.
Maniatis, et al., In: Molecular Cloning, A Laboratory Manual, Cold Spring Harbor Press, NY, 1990.
Matsumoto et al., Int. J. Cancer, 74:482-491, 1997.
McFall and Rapraeger, J. Biol. Chem., 272:12901-12904, 1997.
McFall and Rapraeger, J. Biol. Chem., 273:28270-28276, 1998.
McLean et al., J. Biol. Chem., 265:17126-17131, 1990.
McQuade and Rapraeger, J. Biol. Chem., 278:46607-46615, 2003.
Memmo and McKeown-Longo, J. Cell Sci., 111(Pt 4):425-433, 1998.
Merrifield, Science, 232: 341-347, 1986.
Mertens et al., J. Biol. Chem., 267(28):20435-20443, 1992.
Miranti and Brugge, Nat. Cell Biol., 4:E83-90, 2002.
Mori et al., Cancer Res., 54(13):3396-3397, 1994.
Mould et al., J. Biol. Chem., 271:20365-20374, 1996.
Mundhenke et al., Am. J. Pathol., 160:185-194, 2002.
Myers et al., Am. J. Pathology, 161(6): 2099-2109, 2002.
Myers et al., J. Cell Biol., 148(2): 343-351, 2000.
Nakaerts et al., Int. J. Cancer, 74:335-345, 1997.
Nakanishi et al., Intl. J. Cancer, 80:527-532, 1999.
Nicolau and Sene, Biochim. Biophys. Acta, 721:185-190, 1982.
Nicolau et al., Methods Enzymol., 149:157-176, 1987.
Nobri et al., Nature (London), 368:753-756, 1995.
Noto et al., Int. Immunol., 7:835-842, 1995.
Numa et al., Int. J. Oncol., 20:39-43, 2002.
Oh et al., J. Biol. Chem., 272:11805-11811, 1997b.
Oh et al., J. Biol. Chem., 272:8133-8136, 1997a.
Oh et al., J. Biol. Chem., 273:10624-10629, 1998.
Ohtake et al., Br. J. Cancer, 81:393-403, 1999.
Okamoto etal., Proc. Natl. Acad. Sci. USA, 91(23):11045-11049, 1994.
O'Reilly et al., Cell, 79(2):315-328, 1994.
O'Reilly et al., Cell, 88(2):277-285, 1997.
Orlow et al., Cancer Res, 54(11):2848-2851, 1994.
Ott and Rapraeger, J. Biol. Chem., 273:35291-35298, 1998.
Pampori et al., J. Biol. Chem., 274:21609-21616, 1999.
Panetti et al., J. Biol. Chem., 270:18593-18597, 1995.
Park et al., J. Biol. Chem., 277:29730-29736, 2002.
Pasqualini et al., J. Cell Sci., 105(Pt 1):101-11, 1993.
PCT Appln. WO 94/09699
PCT Appln. WO 95/06128
Petitclerc et al., Cancer Res., 59:2724-2730, 1999.
Pilch et al.,. J. Biol. Chem., 277:21930-21938, 2002.
Plow et al., J. Biol. Chem., 275:21785-21788, 2000.
Potrykus et al., Mol. Gen. Genet., 199:183-188, 1985.
Potter et al., Proc. Natl. Acad. Sci. USA, 81:7161-7165, 1984.
Puklknen et al., Acta Otolaryngol., 117:312-315, 1997.
Rapraeger and Ott, Curr. Opin. Cell Biol., 10:620-628, 1998.
Rapraeger et al., J. Cell Biol., 103:2683-2696, 1986.
Rapraeger, J. Cell Biol., 149:995-998, 2000.
Ratnikov et al., J. Biol. Chem., 277:7377-7385, 2002.
Remington's Pharmaceutical Sciences, 15^{th} ed., pages 1035-1038 and 1570-1580, Mack Publishing Company, Easton, Pa., 1980.
Ridgeway, In: Vectors: A survey of molecular cloning vectors and their uses. Rodriguez and Denhardt (Eds.), Stoneham, Butterworth, 467-492, 1988.
Rintala et al., Gynecol. Onol., 75:372-378, 1999.
Rippe et al., Mol. Cell Biol., 10:689-695, 1990.
Roskelley et al., Curr. Opin. Cell Biol., 7:736-747, 1995.
Sambrook et al., In: Molecular Cloning, A Laboratory Manual, 3rd Ed., Cold Spring Harbor Press, NY, 2001.
Sambrook et al., In: Molecular Cloning, A Laboratory Manual, 3rd Ed., Cold Spring Harbor Press, NY, 2000.
Sanders et al., Cancer Invest., 16:329-344, 1998.
Sanderson and Bernfield, Proc. Natl. Acad. Sci. USA, 85:9562-9566, 1988.
Sanderson and Borset, Ann. Hematol., 81:125-135, 2002.
Sanderson, Semin. Cell Dev. Biol., 12:89-98, 2001.
Saoncella et al., Proc. Natl. Acad. Sci. USA, 96:2805-2810, 1999.
Schaffner-Reckinger et al., J. Biol. Chem., 273:12623-12632, 1998.
Schneller et al., Embo J., 16:5600-5607, 1997.
Serrano et al., Nature, 366:704-707, 1993.
Serrano et al., Science, 267(5195):249-252, 1995.
Shattil et al., J. Cell Biol., 131:807-816, 1995.
Simon et al., J. Biol. Chem., 275:10228-10234, 2000.
Singer et al., J. Cell Biol., 104:573-584, 1987.
Smith et al., J. Biol. Chem., 269:960-967, 1994.
Smith, Matrix Biol., 16:173-178, 1997.
Soldi et al., Embo J., 18:882-892, 1999.
Soukka et al., J. Oral Pathol. Med., 29:308-313, 2000.
Stanley et al., Am. J. Clin. Pathol., 112:377-383, 1999.
Stewart and Young, In: Solid Phase Peptide Synthesis, 2d. Ed., Pierce Chemical Co. 1984.
Streeter and Rees, J. Cell Biol., 105:507-515, 1987.
Stupack et al., J. Cell Biol., 155:459-470,2001.
Sun et al., Int. J. Dev. Biol., 42:733-736, 1998.
Takagi et al., Cell, 110:599-511, 2002.
Tam et al., J. Am. Chem. Soc., 105:6442, 1983.
Timar et al., Am. J. Pathol., 141:467-474, 1992.
Tkachenko and Simons, J. Biol. Chem., 277:19946-19951, 2002.
Tonn et al., Anticancer Res., 18:2599-2605, 1998.
Tsujimoto and Croce, Proc. Natl. Acad. Sci. USA, 83(14):5214-5218, 1986.
Tsujimoto et al., Science, 228(4706):1440-1443, 1985.
Tumova et al., J. Biol. Chem., 275:9410-9417, 2000.
Tur-Kaspa et al., Mol. Cell Biol., 6:716-718, 1986.
van der Flier and Sonnenberg, Cell Tissue Res., 305:285-298, 2001.
van Leeuwen et al., Exp. Dermatol., 5:308-315, 1996.
Vita et al., Biopolymers, 47:93-100, 1998.
Wei et al., Science, 273:1551-1555, 1996.
Weinberg, Science, 254(5035):1138-1146, 1991.
Weisshoff et al., Eur. J. Biochem., 259(3):776-788, 1999.
Wiksten et al., Int. J. Cancer, 95:1-6, 2001.
Wong et al., Gene, 10:87-94, 1980.
Woods and Couchman, Curr. Opin. Cell Biol., 13:578-583, 2001.
Woods and Couchman, Mol. Biol. Cell, 5:183-192, 1994.
Woods et al., Embo J., 5:665-670, 1986.
Wu and Wu, J. Biol. Chem., 262:4429-4432, 1987.
Wu and Wu, Biochemistry, 27:887-892, 1988.
Xue et al., Cancer Res., 57:1682-1689, 1997.
Yamashita et al., J. Immunol., 162:5940-5948, 1999.
Yasuda et al., Int. Immunol., 7:251-258, 1995.

1. An isolated and purified peptide or polypeptide segment consisting of 5-240 amino acid residues and comprising at least 5 contiguous residues of SEQ ID NO:4.

2. The isolated and purified peptide or polypeptide of claim 1, wherein said peptide is 5, 10, 15, 20, 25,30 or 34 amino acid residues in length.

3. The isolated and purified peptide or polypeptide of claim 1, wherein said peptide or polypeptide is 40, 50, 60, 70, 80, 90, 100, 110, 120, 130, 140, 150, 160, 170, 180, 190, 200, 210, 220, 230, 234 or 240 amino acid residues in length.

4. The isolated and purified peptide or polypeptide of claim 1, wherein said peptide or polypeptide comprises 5, 10, 15, 20, 25, 30 or 34 consecutive amino acid residues of SEQ ID NO:4.

5. The isolated and purified peptide of claim 1, wherein said peptide consists of SEQ ID NO:4 or SEQ ID NO:8 or SEQ ID NO:9.

6. The isolated and purified peptide or polypeptide of claim 1, wherein said peptide or polypeptide comprises 5, 10, 15, 20, 25, 30, 34, 40, 50, 60, 70, 80, 90, 100, 110, 120, 130, 140, 150, 160, 170, 180, 190, 200, 210, 220, 230, 234 or 240 consecutive amino acid residues of SEQ ID NO:8.

7. The isolated and purified peptide of claim 1, wherein said peptide consists of SEQ ID NO:8.

8. A nucleic acid encoding an isolated and purified peptide or polypeptide consisting of 5-240 amino acid residues and comprising at least 5 contiguous residues of SEQ ID NO:4 or SEQ ID NO:8 or SEQ ID NO:9.

9. The nucleic acid of claim 8, wherein said nucleic acid encodes a peptide or Ipolypeptide of 5, 10, 15, 20, 25, 30, 34, 40, 50, 60, 70, 80, 90, 100, 110, 120, 130, 140, 150, 160, 170, 180, 190, 200, 210, 220, 230, 234 or 240 amino acid residues in length.

10. The nucleic acid of claim 8, wherein said nucleic acid encodes a peptide or polypeptide comprising 5, 10, 15, 20, 25, 30, 34, 40, 50, 60, 70, 80, 90, 100, 110, 120, 130, 140,150, 160, 170, 180, 190, 200, 210, 220, 230, 234 or 240 consecutive amino acid residues of SEQ ID NO:4 or SEQ ID NO:8 or SEQ ID NO:9.

11.-13. (canceled)

14. A method of inhibiting interaction of αᵥβ₃ or αᵥβ₅ integrin with syndecan-1 comprising contacting a αᵥβ₃or αᵥβ₅ integrin molecule with a peptide or polypeptide consisting of 5-240 amino acid residues and comprising at least 5 contiguous residues of SEQ ID NO:4 or SEQ ID NO:8 or SEQ ID NO:9.

15. The method of claim 14, wherein said peptide or polypeptide is 5, 10, 15, 20, 25, 30, 34, 40, 50, 60, 70, 80, 90, 100, 110, 120, 130, 140, 150, 160, 170, 180,190, 200, 210, 220, 230, 234 or 240 amino acid residues in length.

16. The method of claim 14, wherein said peptide or polypeptide comprises 5, 10, 15, 20, 25, 30, 34,40, 50, 60, 70, 80, 90, 100, 110, 120, 130, 140, 150, 160, 170, 180, 190, 200, 210, 220, 230, 234 or 240 consecutive amino acid residues of SEQ ID NO:4 or SEQ ID NO:8 or SEQ ID NO:9.

17. The method of claim 14, wherein said peptide consists of SEQ ID NO:4 or SEQ ID NO:8 or SEQ ID NO:9.

18. The method of claim 14, wherein said αᵥβ₃ or αᵥβ₅ integrin is located on the surface of a cell.

19. A method of inhibiting αᵥβ₃ or αᵥβ₅ integrin activation by syndecan-1 comprising contacting a cell expressing an αᵥβ₃ or αᵥβ₅ integrin molecule with a peptide or polypeptide consisting of 5-240 amino acid residues and comprising at least 5 contiguous residues of SEQ ID NO:4 or SEQ ID NO:8 or SEQ ID NO:9.

20. A method of inhibiting a cancer cell expressing αᵥβ₃ or or αᵥβ₅ integrin comprising contacting a cell expressing an αᵥβ₃ or or αᵥβ₅ integrin molecule with a peptide or polypeptide consisting of 5-240 amino acid residues and comprising at least 5 contiguous residues of SEQ ID NO:4 or SEQ ID NO:8 or SEQ ID NO:9.

21. The method of claim 20, wherein said peptide or polypeptide is 5, 10, 15, 20, 25, 30, 34, 40, 50, 60, 70, 80, 90, 100, 110, 120, 130, 140, 150, 160, 170, 180, 190, 200, 210, 220, 230, 234 or 240 amino acid residues in length.

22. The method of claim 20, wherein said peptide or polypeptide comprises 5, 10, 15, 20, 25, 30, 34, 40, 50, 60, 70, 80, 90, 100, 110, 120, 130, 140, 150, 160, 170, 180, 190, 200, 210, 220, 230, 234 or 240 consecutive amino acid residues of SEQ ID NO:4 or SEQ ID NO:8 or SEQ ID NO:9.

23. The method of claim 20, wherein inhibiting comprises inhibiting migration, metastasis, survival and/or proliferation.

24. The method of claim 20, wherein said cancer cell is a carcinoma, a myeloma, a melanoma or a glioma.

25. The method of claim 20, further comprising contacting said cell with a second cancer inhibitory agent.

26. A method of treating a subject with a cancer, cells of which express αᵥβ₃or or αᵥβ₅ integrin, comprising contacting a cell expressing an αᵥβ₃ or or αᵥβ₅ integrin molecule with a peptide or polypeptide consisting of 5-240 amino acid residues and comprising at least 5 contiguous residues of SEQ ID NO:4 or SEQ ID NO:8 or SEQ ID NO:9.

27. The method of claim 26, wherein said peptide or polypeptide is 5, 10, 15, 20, 25, 30, 34, 40, 50, 60, 70, 80, 90, 100, 110, 120, 130, 140, 150, 160, 170, 180, 190, 200, 210, 220, 230, 234 or 240 amino acid residues in length.

28. The method of claim 26, wherein said peptide or polypeptide comprises 5, 10, 15, 20, 25, 30, 34, 40, 50, 60, 70, 80, 90, 100, 110, 120, 130, 140, 150, 160, 170, 180, 190, 200, 210, 220, 230, 234 or 240 consecutive amino acid residues of SEQ ID NO:4 or SEQ ID NO:8 or SEQ ID NO:9.

29. The method of claim 28, wherein said subject is a human.

30. The method of claim 28, wherein said cancer is a carcinoma, a myeloma, a melanoma or a glioma.

31. The method of claim 28, wherein said peptide or polypeptide is administered directly to said cancer cells, local to said cancer cells, regional to said cancer cells, or systemically.

32. The method of claim 28, further comprising administering to said subject a second cancer therapy selected from chemotherapy, radiotherapy, immunotherapy, hormonal therapy, or gene therapy.

33.-35. (canceled)
